(19) United States
(12) Patent Application Publication
(10) Pub. No.: US 2024/0425805 A1
(43) Pub. Date: Dec. 26, 2024

(54) CELL CULTURE METHODS

(71) Applicant: INTERNATIONAL N&H DENMARK APS, KONGENS LYNGBY (DK)

(72) Inventors: Ritesh Kumar, Wilmington, DE (US); Helene Kane, Wilmington, DE (US); Charles Budinoff, Oregon, WI (US)

(21) Appl. No.: 18/569,795

(22) PCT Filed: Jun. 29, 2022
(86) PCT No.: PCT/US2022/035530

§ 371 Date: Dec. 13, 2023

Related U.S. Application Data
(60) Provisional application No. 63/335,419, filed on Apr. 27, 2022.
(60) Provisional application No. 63/330,693, filed on Apr. 13, 2022.
(60) Provisional application No. 63/216,193, filed on Jun. 29, 2021.

(51) Int. Cl. C12N 1/20; A23K 10/16; C12N 1/16; C12N 1/38; C12N 5/00; C12N 5/071; C12N 9/16; C12R 1/225; C12R 1/46

(52) U.S. Cl. CPC C12N 1/20; A23K 10/16; C12N 1/16; C12N 1/38; C12N 5/0018; C12N 5/0686; C12N 9/16; C12N 2500/40; C12N 2501/73; C12R 2001/225; C12R 2001/46; C12Y 301/03001; C12Y 301/03002; C12Y 306/01005

(57) ABSTRACT

Provided herein, inter alia, are methods for improving the growth of cells (such as probiotic cells or recombinant cells) by culturing the cells in a liquid media comprising one or more enzymes that hydrolyze at least one nucleoside triphosphate present in the media. Cells cultured in accordance with these methods exhibit improved growth compared to cells cultured in media that does not comprise one or more enzymes that hydrolyze at least one nucleoside triphosphate.

### CROSS-REFERENCE TO RELATED APPLICATIONS

This application is a 371 of International Application No. PCT/US2022/035530, filed Jun. 29, 2022 and claims priority to U.S. Provisional Patent Application No. 63/216,193, filed Jun. 29, 2021, U.S. Provisional Patent Application No. 63/330,693, filed Apr. 13, 2022, and U.S. Provisional Patent Application No. 63/335,419, filed Apr. 27, 2022, the disclosure of each of which is incorporated by reference herein in its entirety.

### SEQUENCE LISTING

The official copy of the sequence listing is submitted electronically via Patent Center as an ASCII formatted sequence listing with a file named 20231208_NB41935USPCT_Seq_List_ST25 created on Dec. 8, 2023 and having a size of 23,855 bytes and is filed concurrently with the specification. The sequence listing contained in this ASCII formatted document is part of the specification and is herein incorporated by reference in its entirety.

### FIELD OF THE INVENTION

Provided herein, inter alia, are improved methods for culturing cells in liquid media.

### BACKGROUND

Before being inoculated into probiotic-based products, such as food products, animal feeds, or dietary supplements, microorganisms are cultured in liquid media to provide a suspension containing large amounts of the microorganisms. This suspension is usually then concentrated using centrifugation, filtration, distillation, sedimentation or flocculation. This concentration step is often followed by freezing or freeze-drying or drying or storage of the microorganism concentrate as a frozen product to preserve and/or store the microorganism.

A limiting step in the production of such probiotic-based products is the quantity and viability of microorganisms capable of being produced in liquid culture. As such, and given the ever-increasing demand for such products, there remains a need to improve the efficiency of methods for producing microorganism-containing suspensions in liquid culture to obtain highly concentrated microorganism suspensions with limited loss of biological activity as well as limited loss of viable microorganisms both during production and subsequent processing and storage. These methods need to be feasible at any scale, but especially on the industrial scale, where large volumes of suspension are concentrated.

The subject matter disclosed herein addresses this need and provides additional benefits as well.

### SUMMARY

Provided herein, inter alia, are methods for improving the growth of cells cultured in a liquid media. The methods require culturing the cells in a liquid media comprising one or more enzymes that hydrolyze at least one nucleoside triphosphate present in the media. Cells cultured in accordance with these methods exhibit improved growth compared to cells cultured in media that does not comprise one or more enzymes that hydrolyze at least one nucleoside triphosphate.

Accordingly, in some aspects, provided herein are methods for improving the growth of cells cultured in a liquid media comprising culturing the cells in a media comprising one or more enzymes that hydrolyze at least one nucleoside triphosphate, wherein the cells exhibit improved growth compared to cells cultured in media that does not comprise one or more enzymes that hydrolyze at least one nucleoside triphosphate. In some embodiments, improved growth comprises one or more of greater optical density (OD), higher colony forming units (cfu)/mL, decreased cell lysis, higher proliferation rate, increased cell mass, higher cell numbers, or higher post-culture viability. In some embodiments of any of the embodiments disclosed herein, the nucleoside triphosphate comprises one or more of ATP, GTP, CTP, TTP, and/or UTP. In some embodiments, the nucleoside triphosphate comprises ATP. In some embodiments of any of the embodiments disclosed herein, the enzyme is one or more phosphatase. In some embodiments, the phosphatase is one or more of an acid phosphatase, an alkaline phosphatase, or an apyrase. In some embodiments of any of the embodiments disclosed herein, the cultured cells are bacterial, fungal, archaeal, plant, insect, or mammalian cells. In some embodiments, the cells are recombinant cells. In some embodiments of any of the embodiments disclosed herein, the bacterial cells are gram positive or gram negative. In some embodiments, the bacterial cells are aerobes or anaerobes. In some embodiments of any of the embodiments disclosed herein, the bacterial cells are probiotics. In some embodiments of any of the embodiments disclosed herein, the fungal cells are filamentous fungal cells. In some embodiments of any of the embodiments disclosed herein, the fungal cells are yeast cells. In some embodiments of any of the embodiments disclosed herein, the mammalian cells are primary cells, Chinese hamster ovary (CHO) cells or human embryonic kidney (HEK) cells. In some embodiments of any of the embodiments disclosed herein, the recombinant cells produce an enzyme. In some embodiments of any of the embodiments disclosed herein, the recombinant cells produce an antibody. In some embodiments of any of the embodiments disclosed herein, the primary cell is a stem cell or an immune cell. In some embodiments, the immune cell is selected from the group consisting of: a natural killer (NK) cell, a lymphocyte, a leukocyte, and a monocyte. In some embodiments of any of the embodiments disclosed herein, the one or more enzymes that hydrolyze at least one nucleoside triphosphate is exogenously added to the media. In some embodiments of any of the embodiments disclosed herein, the one or more enzymes that hydrolyze at least one nucleoside triphosphate is not recombinantly or naturally produced by cells in the liquid media. In some embodiments of any of the embodiments disclosed herein, the one or more enzyme that hydrolyzes at least one nucleoside triphosphate comprises one or more enzyme at least about 60% identical to the amino acid sequence of SEQ ID NOs: 1, 2, 3, 4, 5 and/or 6.

In further aspects, provided herein is a method for culturing a probiotic for consumption by a subject, the method comprising culturing probiotic cells in a liquid media comprising one or more enzymes that hydrolyze at least one nucleoside triphosphate, wherein the probiotic cells exhibit improved growth compared to probiotic cells cultured in media that does not comprise one or more enzymes that hydrolyze at least one nucleoside triphosphate. In some embodiments, improved growth comprises one or more of greater optical density (OD), higher colony forming units (cfu)/mL, decreased cell lysis, higher proliferation rate, increased cell mass, higher cell numbers, or higher post-culture viability. In some embodiments of any of the embodiments disclosed herein, the nucleoside triphosphate comprises one or more of ATP, GTP, CTP, TTP, and/or UTP. In some embodiments, the nucleoside triphosphate comprises ATP. In some embodiments of any of the embodiments disclosed herein, the enzyme is one or more phosphatase. In some embodiments, the phosphatase is one or more of an acid phosphatase, an alkaline phosphatase, or an apyrase. In some embodiments of any of the embodiments disclosed herein, the probiotic cells are bacterial or fungal cells. In some embodiments, the cells are recombinant cells. In some embodiments of any of the embodiments disclosed herein, the bacterial cells are gram positive or gram negative. In some embodiments, the bacterial cells are aerobes or anaerobes. In some embodiments of any of the embodiments disclosed herein, the fungal cells are yeast cells. In some embodiments of any of the embodiments disclosed herein, the recombinant cells produce an enzyme. In some embodiments of any of the embodiments disclosed herein, the method further comprises harvesting the probiotic cells. In some embodiments of any of the embodiments disclosed herein, the method further comprises freeze drying or lyophilizing the probiotic cells. In some embodiments of any of the embodiments disclosed herein, the subject is a human, a companion animal, or livestock. In some embodiments, the livestock is poultry, swine, or a ruminant animal. In some embodiments of any of the embodiments disclosed herein, the method further comprises formulating the probiotic cells into a dosage form suitable for enteral administration. In some embodiments of any of the embodiments disclosed herein, the method further comprises combining the probiotic cells with an animal feed. In some embodiments of any of the embodiments disclosed herein, the method further comprises administering the probiotic cells to the subject via a waterline. In some embodiments of any of the embodiments disclosed herein, the one or more enzymes that hydrolyze at least one nucleoside triphosphate is exogenously added to the media. In some embodiments of any of the embodiments disclosed herein, the one or more enzymes that hydrolyze at least one nucleoside triphosphate is not recombinantly or naturally produced by the probiotic cells in the liquid media. In some embodiments of any of the embodiments disclosed herein, the one or more enzyme that hydrolyzes at least one nucleoside triphosphate comprises one or more enzyme at least about 60% identical to the amino acid sequence of SEQ ID NOs: 1, 2, 3, 4, 5 and/or 6.

In another aspect, provided herein is a method for producing bacterial cells for use in production of fermented dairy products, the method comprising culturing the bacterial cells in a liquid media comprising one or more enzymes that hydrolyze at least one nucleoside triphosphate, wherein the bacterial cells exhibit improved growth compared to bacterial cells cultured in media that does not comprise one or more enzymes that hydrolyze at least one nucleoside triphosphate. In some embodiments, improved growth comprises one or more of greater optical density (OD), higher colony forming units (cfu)/mL, decreased cell lysis, higher proliferation rate, increased cell mass, higher cell numbers, or higher post-culture viability. In some embodiments of any of the embodiments disclosed herein, the nucleoside triphosphate comprises one or more of ATP, GTP, CTP, TTP, and/or UTP. In some embodiments, the nucleoside triphosphate comprises ATP. In some embodiments of any of the embodiments disclosed herein, the enzyme is one or more phosphatase. In some embodiments, the phosphatase is one or more of an acid phosphatase, an alkaline phosphatase, or an apyrase. In some embodiments of any of the embodiments disclosed herein, the bacterial cells are gram positive or gram negative. In some embodiments, the bacterial cells are thermophilic. In some embodiments of any of the embodiments disclosed herein, the bacterial cells are selected from the group consisting of a Lactobacillus spp., a Streptococcus spp., and a Lactococcus spp. In some embodiments of any of the embodiments disclosed herein, the fermented dairy product is a yoghurt, a cream, a matured cream, a cheese, fromage frais, a milk beverage, a processed cheese, a cream dessert, a cottage cheese, an infant milk, or a kefir. In some embodiments of any of the embodiments disclosed herein, the one or more enzymes that hydrolyze at least one nucleoside triphosphate is exogenously added to the media. In some embodiments of any of the embodiments disclosed herein, the one or more enzymes that hydrolyze at least one nucleoside triphosphate is not recombinantly or naturally produced by the bacterial cells in the liquid media. In some embodiments of any of the embodiments disclosed herein, the one or more enzyme that hydrolyzes at least one nucleoside triphosphate comprises one or more enzyme at least about 60% identical to the amino acid sequence of SEQ ID NOs: 1, 2, 3, 4, 5 and/or 6.

In yet other aspects, provided herein is a culture media comprising one or more enzymes that hydrolyze at least one nucleoside triphosphate, wherein said one or more enzymes that hydrolyze at least one nucleoside triphosphate is a) recombinantly produced; and b) exogenously added to the media. In some embodiments, the media does not comprise one or more cells. In some embodiments, the media comprises one or more cells and wherein said one or more cells do not recombinantly produce one or more enzymes that hydrolyze at least one nucleoside triphosphate. In some embodiments of any of the embodiments disclosed herein, the nucleoside triphosphate comprises one or more of ATP, GTP, CTP, TTP, and/or UTP. In some embodiments, the nucleoside triphosphate comprises ATP. In some embodiments of any of the embodiments disclosed herein, the enzyme is one or more phosphatase. In some embodiments, the phosphatase is one or more of an acid phosphatase, an alkaline phosphatase, or an apyrase. In some embodiments of any of the embodiments disclosed herein, the cells are bacterial, fungal, archaeal, plant, insect, or mammalian cells. In some embodiments, the cells are recombinant cells. In some embodiments of any of the embodiments disclosed herein, the bacterial cells are gram positive or gram negative. In some embodiments of any of the embodiments disclosed herein, the bacterial cells are aerobes or anaerobes. In some embodiments of any of the embodiments disclosed herein, the bacterial cells are probiotics. In some embodiments of any of the embodiments disclosed herein, the fungal cells are filamentous fungal cells. In some embodiments of any of the embodiments disclosed herein, the fungal cells are yeast cells. In some embodiments of any of the embodiments disclosed herein, the mammalian cells are primary cells, Chinese hamster ovary (CHO) cells or human embryonic kidney (HEK) cells. In some embodiments of any of the embodiments disclosed herein, the recombinant cells produce an enzyme. In some embodiments of any of the embodiments disclosed herein, the recombinant cells produce an antibody. In some embodiments of any of the embodiments disclosed herein, the primary cell is a stem cell or an immune cell. In some embodiments of any of the embodiments disclosed herein, the immune cell is selected from the group consisting of: a natural killer (NK) cell, a lymphocyte, a leukocyte, and a monocyte. In some embodiments of any of the embodiments disclosed herein, the one or more enzyme that hydrolyzes at least one nucleoside triphosphate comprises one or more enzyme at least about 60% identical to the amino acid sequence of SEQ ID NOs: 1, 2, 3, 4, 5 and/or 6.

In another aspect, provided herein is a method for producing a liquid cell culture media comprising combining a cell culture media with one or more recombinant enzymes that hydrolyze at least one nucleoside triphosphate. In some embodiments, the nucleoside triphosphate comprises one or more of ATP, GTP, CTP, TTP, and/or UTP. In some embodiments, the nucleoside triphosphate comprises ATP. In some embodiments of any of the embodiments disclosed herein, the enzyme is one or more phosphatase. In some embodiments, the phosphatase is one or more of an acid phosphatase, an alkaline phosphatase, or an apyrase. In some embodiments of any of the embodiments disclosed herein, the one or more recombinant enzyme that hydrolyzes at least one nucleoside triphosphate comprises one or more enzyme at least about 60% identical to the amino acid sequence of SEQ ID NOs: 1, 2, 3, 4, 5 and/or 6.

In still other aspects, provided herein is A method for improving the growth of cells engineered to produce a first recombinant protein comprising culturing the cell in a media comprising one or more enzymes that hydrolyze at least one nucleoside triphosphate, wherein 1) the cells exhibit improved growth compared to cells cultured in media that does not comprise one or more enzymes that hydrolyze at least one nucleoside triphosphate; and 2) wherein the one or more enzymes that hydrolyze at least one nucleoside triphosphate is a) exogenously added to the media; and/or b) recombinantly expressed by the engineered cells in addition to the first recombinant protein. In some embodiments, improved growth comprises one or more of greater optical density (OD), higher colony forming units (cfu)/mL, decreased cell lysis, higher proliferation rate, increased cell mass, higher cell numbers, or higher recombinant protein production. In some embodiments of any of the embodiments disclosed herein, the nucleoside triphosphate comprises one or more of ATP, GTP, CTP, TTP, and/or UTP. In some embodiments, the nucleoside triphosphate comprises ATP. In some embodiments of any of the embodiments disclosed herein, the enzyme is one or more phosphatase. In some embodiments, the phosphatase is one or more of an acid phosphatase, an alkaline phosphatase, or an apyrase. In some embodiments of any of the embodiments disclosed herein, the cultured cells are bacterial, fungal, archaeal, plant, insect, or mammalian cells. In some embodiments, the bacterial cells are gram positive or gram negative. In some embodiments, the bacterial cells are aerobes or anaerobes. In some embodiments, the fungal cells are filamentous fungal cells. In some embodiments, the fungal cells are yeast cells. In some embodiments, the mammalian cells are primary cells, Chinese hamster ovary (CHO) cells or human embryonic kidney (HEK) cells. In some embodiments of any of the embodiments disclosed herein, the recombinant cells produce an enzyme. In some embodiments of any of the embodiments disclosed herein, the recombinant cells produce an antibody. In some embodiments of any of the embodiments disclosed herein, the primary cell is a stem cell or an immune cell. In some embodiments, the immune cell is selected from the group consisting of: a natural killer (NK) cell, a lymphocyte, a leukocyte, and a monocyte. In some embodiments of any of the embodiments disclosed herein, one or more polynucleotides encoding the one or more enzymes that hydrolyze at least one nucleoside triphosphate is recombinantly expressed on one or more extrachromosomal vector in the cell. In some embodiments of any of the embodiments disclosed herein, one or more polynucleotides encoding the one or more enzymes that hydrolyze at least one nucleoside triphosphate is recombinantly expressed via stable integration into the chromosome of the cell. In some embodiments of any of the embodiments disclosed herein, the one or more enzymes that hydrolyze at least one nucleoside triphosphate is recombinantly expressed by the engineered cells in addition to the first recombinant protein and is further engineered to be secreted by the cell. In some embodiments of any of the embodiments disclosed herein, the one or more enzyme that hydrolyzes at least one nucleoside triphosphate comprises one or more enzyme at least about 60% identical to the amino acid sequence of SEQ ID NOs: 1, 2, 3, 4, 5 and/or 6.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 depicts a graph illustrating comparative growth over time as measured by OD₆₀₀ of B. animalis in liquid culture media in the presence of exogenously added ATP (diamonds), exogenously added ATP and apyrase (circles), and exogenously added apyrase (triangles) compared to cell-only control (squares).

FIG. 2 depicts a bar graph illustrating ATP presence (nM) in liquid media over time in the presence and absence of exogenously added apyrase.

FIG. 3 depicts a graph illustrating comparative growth over time as measured by OD₆₀₀ of B. animalis in liquid culture media in the presence of exogenously added apyrase (squares) and exogenously added heat-killed apyrase (triangles) compared to cell-only control (circles).

FIG. 4A, FIG. 4B, and FIG. 4C depict the effect of ATPase on CHO cell growth.

FIG. 4A shows effect of added potato apyrase. FIG. 4B shows effect of added CRC22110 phosphatase. Cell count was determined after 60 hours of culture. FIG. 4C depicts the effect on cell viability of CHO cell cultures in the presence of added potato apyrase.

FIG. 5 depicts the effect of exogenously added apyrase on detectable GFP expression of CHO-GFP cell line in adherent culture.

### DETAILED DESCRIPTION

Adenosine triphosphate (ATP) is one of the most important indicators of cell viability. Extracellular ATP (eATP) is commonly detected in cultures of both eukaryotic and prokaryotic cells but has not been studied as a modulator of cell growth. Although ATP release has traditionally been considered mainly to occur because of cell lysis and apoptosis, current evidence indicates that ATP leakage can also occur during the growth phase of diverse microbial species and may play an important role in bacterial physiology (Spari & Beldi, Int J Mol Sci. 2020 Aug. 4; 21 (15): 5590). While eATP has been shown to modulate signaling in eukaryotic cells, the role of eATP in prokaryote cell signaling has not been well studied.

When cells are grown in suspension, they experience stress. Without being bound to theory, this stress could contribute to or be responsible for apoptosis and cell death. Cell lysis following apoptosis results in the release of ATP, further enhancing apoptosis in the cell culture as well as impacting cell health and productivity in terms of protein production. The inventors of the instant application have surprisingly found that the growth of several microbial species is sensitive to the presence of eATP in liquid culture media and that addition of an enzyme capable of hydrolyzing this nucleoside triphosphate can negate the negative effects of ATP on cell growth and/or viability. Thus, the current invention provides a novel way to overcome production constraints in prokaryotic, fungal, eukaryotic, and insect cell line-based production platforms.

As shown in the Examples section, the inventors have shown that numerous species of microorganisms release ATP in a growth-dependent manner during cell culture using multiple examples of liquid media and that this released eATP can suppress cell growth. Further, it is shown that addition of any enzyme capable of hydrolyzing ATP can reverse the negative effect of eATP on microbial growth, thereby resulting in enhanced growth yields and/or viable cells.

### I. Definitions

“Nucleoside triphosphate,” as used herein, refers to molecules containing a nitrogenous base bound to a 5-carbon sugar (either ribose or deoxyribose) with three phosphate groups bound to the sugar. Non-limiting examples of nucleoside triphosphates include, for example adenosine triphosphate (ATP), deoxyribose adenosine triphosphate (dATP), guanine triphosphate (GTP), deoxyribose guanine triphosphate (dGTP), cytosine triphosphate (CTP), deoxyribose cytosine triphosphate (dCTP), deoxyribose thymine triphosphate (dTTP), and uracil triphosphate (UTP).

The phrase “enzyme that hydrolyzes at least one nucleoside triphosphate” refers to any molecule that can cleave at least the third (i.e. outermost with respect to the location of the pentose base of the nucleoside triphosphate) phosphate of a nucleoside triphosphate. Such enzymes include, without limitation, phosphatases.

A “phosphatase” is used synonymously with the term “NTPase” to refer to an enzyme that removes a phosphate group from its substrate by hydrolyzing phosphoric acid monoesters into a phosphate ion and a molecule with a free hydroxyl group. Exemplary phosphatases include alkaline phosphatases, acid phosphatases, or apyrases.

An “acid phosphatase” (EC 3.1.3.2), as used herein, refers to any phosphatase derived from any source that optimally catalyzes the hydrolysis of phosphate esters in an acidic environment (i.e. with a pH<7).

An “alkaline phosphatase” (EC 3.1.3.1), as used herein, refers to any phosphatase derived from any source that catalyzes the hydrolysis of phosphate esters in an alkaline environment (i.e. with a pH>7).

An “apyrase” (EC 3.6.1.5), refers to any enzyme that catalyzes the hydrolysis of NTP to yield NMP and inorganic phosphate. Apyrase can also act on NDP and other nucleoside triphosphates and diphosphates with the general reaction being NTP−>NDP+Pi−>NMP+2Pi.

The phrase “improving growth of cells cultured in a liquid media” or “improved growth” refers to improving the yield or viability of any cell cultured or fermented in a nutrient-rich liquid medium over a given period of time. Non-limiting examples of cell growth improvement include greater post-culture optical density (OD), higher post-culture colony forming units (cfu)/mL, decreased cell lysis, higher proliferation rate, increased cell mass, higher cell numbers, or higher post-culture viability.

As used herein, “microorganism” or “microbe” refers to a bacterium, a fungus, a protozoan, an alga, an archaeon, and other microbes or microscopic organisms. Furthermore, the term “microorganism” also encompasses additional eukaryotic cells (i.e., eukaryotic cells other than fungal and algal cells, for example yeast, insect (such as, without limitation, Drosophila S2 cells), mammalian cells or mammalian cell lines (such as, without limitation, Chinese hamster ovary (CHO) cells, human embryonic kidney (HEK) cells, stem cells, or primary cells)) as well as plant cells).

As used here in the term “probiotic” refers to a composition for consumption by subjects (for example, humans) that contains viable (i.e. live) microorganisms, i.e. microorganisms that are capable of living and reproducing that, when administered in adequate amounts, confer a health benefit on a subject (see Hill et al. 2014 Nature Revs Gastro & Hep 11, 506-514, incorporated by reference herein in its entirety). Probiotics are distinguished from bacterial compositions that have been killed, for example, by pasteurization or heat treatment. Compositions comprising non-viable microorganisms are also contemplated in certain embodiments of the methods disclosed herein.

A “strain” as used herein refers to a microorganism which remains genetically unchanged when grown or multiplied. The multiplicity of identical microorganisms is included.

By “at least one strain,” is meant a single strain but also mixtures of strains comprising at least two strains of microorganisms. By “a mixture of at least two strains,” is meant a mixture of two, three, four, five, six or even more strains. In some embodiments of a mixture of strains, the proportions can vary from 1% to 99%. When a mixture comprises more than two strains, the strains can be present in substantially equal proportions in the mixture or in different proportions.

For purposes of this disclosure, a “biologically pure strain” means a strain containing no other bacterial strains in quantities sufficient to interfere with replication of the strain or to be detectable by normal bacteriological techniques. “Isolated” when used in connection with the organisms and cultures described herein includes not only a biologically pure strain, but also any culture of organisms which is grown or maintained other than as it is found in nature. In some embodiments, the strains are mutants, variants, or derivatives of strains.

The term “primary cell” as used herein is known in the art to refer to a cell that has been isolated from a tissue and has been established for growth in vitro. Corresponding cells have undergone very few, if any, population doublings and are therefore more representative of the main functional component of the tissue from which they are derived in comparison to continuous cell lines thus representing a more representative model to the in vivo state. Methods to obtain samples from various tissues and methods to establish primary cell lines are well-known in the art. Non-limiting examples of primary cells include stem cells and immune cells.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

The term “immune cells” as used herein refers to cells that are part of the immune system (which can be either the adaptive or the innate immune system). Immune cells can include white blood cells (leukocytes), including lymphocytes, monocytes, macrophages and dendritic cells. Lymphocytes can include T cells, natural killer (NK) cells and B cells. Immune cells as used herein can be those that are manufactured for adoptive cell transfer (either autologous transfer or allogeneic transfer). In the context of adoptive transfer, note that immune cells can be primary cells (i.e. cells isolated directly from human or animal tissue, and not or only briefly cultured) but can also be cell lines (i.e. cells that have been continually passaged over a long period of time and have acquired homogenous genotypic and phenotypic characteristics).

As used herein, “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism is recombinant, for example, if it is synthesized from an mRNA synthesized from a recombinant gene present in the cell.

As used herein, “recombinant cell,” “genetically engineered cell,” and “synthetic cell” are used interchangeably to refer to a cell that has been genetically altered to express one or more nucleic acid sequences. The cell may or may not also endogenously express the same nucleic acid sequences.

The term “subject” as used herein includes all non-ruminant (including mammals, for example, humans) and ruminant animals. In one embodiment, the subject is a non-ruminant subject and a monogastric subject, such as a human. Additional examples of monogastric subjects include, but are not limited to, pigs and swine, such as piglets, growing pigs, and sows; poultry, such as emu, pheasant, grouse, turkeys, ducks, chicken, broiler chicks, and layers; fish, such as salmon, trout, tilapia, catfish, mahi mahi, and carp; and crustaceans such as shrimp and prawns. In a further embodiment the subject is a ruminant animal including, but not limited to, cattle, young calves, goats, sheep, giraffes, bison, moose, elk, yaks, water buffalo, deer, camels, alpacas, llamas, antelope, pronghorn and nilgai.

As used herein “administer” or “administering” is meant the action of introducing one or more compositions comprising one or more microorganism (such as one or more microorganism strains), to a subject, such as by feeding or consuming enterally (such as orally or rectally). The composition containing one or more microbial strains can also be administered in one or more doses.

“Enteral administration,” as used herein, means administration via absorption through the oral, buccal, sublingual and/or gastrointestinal tract. Enteral administration can include oral and sublingual administration, gastric administration, or rectal administration.

“Fermented dairy products,” as used herein, refer to products intended for consumption, by humans or animals, which are derived by fermentation and by the use of a lactic acid bacterium. As a non-limiting example, “fermented dairy product” refers to product selected from the group consisting of yogurt, cheese and fermented milk products.

As used herein, “freeze-drying” (and other forms thereof, such as lyophilization) refers to any process by which water is removed from a material which is first frozen and then subjected to reduced pressure and/or heat which allows the water to sublime directly from the solid phase to gas.

As used herein, “exogenously added,” molecules such as enzymes (for example, an enzyme that hydrolyzes at least one nucleoside triphosphate) and the like, in the context of cell culture or liquid media, refers to molecules that are added to the cell culture or liquid media and which are not recombinantly or naturally produced by the cells in the cell culture or liquid media.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Compositions and Methods

Provided herein are compositions and methods for improving the growth of cells cultured in a liquid media. In one embodiment, the method comprises culturing the cells in a media containing one or more enzymes (for example, one or more exogenously added enzymes) that hydrolyze at least one nucleoside triphosphate. The cultured cells exhibit improved growth compared to cells cultured in media that does not comprise one or more enzymes that hydrolyze at least one nucleoside triphosphate.

A. Enzymes that Hydrolyze at Least One Nucleoside Triphosphate

Any enzyme capable of removing the third phosphate of a nucleoside triphosphate obtainable from any source organism can be used in the cell culture methods and compositions disclosed herein. In some embodiments, the enzyme is a phosphatase, falling within the EC 3.1.3.x (where x is an integer from 1 to 86, i.e., phosphoric monoester hydrolases). These enzymes include, without limitation, those falling within the following enzyme classification numbers: EC 3.1.3.1 alkaline phosphatase, EC 3.1.3.2 acid phosphatase, EC 3.1.3.3 phosphoserine phosphatase, EC 3.1.3.4 phosphatidate phosphatase, EC 3.1.3.5 5′-nucleotidase, EC 3.1.3.6 3′-nucleotidase, EC 3.1.3.7 3′ (2′),5′-bisphosphate nucleotidase, or EC 3.1.3.8 3-phytase. In other embodiments, the phosphatase is EC 3.6.1.5 apyrase (for example, potato apyrase).

In some embodiments, the enzyme capable of removing the third phosphate of a nucleoside triphosphate comprises an amino acid sequence at least about 60% identical to SEQ ID NO:1 (such as any of about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:1).

In some embodiments, the enzyme capable of removing the third phosphate of a nucleoside triphosphate comprises an amino acid sequence at least about 60% identical to SEQ ID NO:2 (such as any of about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:2).

In some embodiments, the enzyme capable of removing the third phosphate of a nucleoside triphosphate comprises an amino acid sequence at least about 60% identical to SEQ ID NO:3 (such as any of about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:3).

In some embodiments, the enzyme capable of removing the third phosphate of a nucleoside triphosphate comprises an amino acid sequence at least about 60% identical to SEQ ID NO:4 (such as any of about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:4).

In some embodiments, the enzyme capable of removing the third phosphate of a nucleoside triphosphate comprises an amino acid sequence at least about 60% identical to SEQ ID NO:5 (such as any of about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:5).

In some embodiments, the enzyme capable of removing the third phosphate of a nucleoside triphosphate comprises an amino acid sequence at least about 60% identical to SEQ ID NO:6 (such as any of about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:6).

The phosphatase can be added to a liquid cell culture media, such as any of the exemplary liquid cell culture media disclosed herein in a concentration from about 5 nM to about 500 nM, such as any of about 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, 300 nM, 310 nM, 320 nM, 330 nM, 340 nM, 350 nM, 360 nM, 370 nM, 380 nM, 390 nM, 400 nM, 410 nM, 420 nM, 430 nM, 440 nM, 450 nM, 460 nM, 470 nM, 480 nM, 490 nM, or 500 nM or more, inclusive of all concentrations falling in between these values.

### B. Exemplary Cells

Any cell, whether bacterial, fungal, archaeal, plant, insect, or mammalian, can be cultured in liquid media with one or more enzymes that hydrolyze at least one nucleoside triphosphate in accordance with the methods disclosed herein.

In some embodiments, the cell is a fungus, examples of which are species of Aspergillus such as A. oryzae and A. niger, species of Saccharomyces such as S. cerevisiae, species of Schizosaccharomyces such as S. pombe, and species of Trichoderma such as T. reesei.

In some embodiments, the cell is a filamentous fungal cell. The term “filamentous fungi” refers to all filamentous forms of the subdivision Eumycotina (see, Alexopoulos, C. J. (1962), Introductory Mycology, Wiley, New York). These fungi are characterized by a vegetative mycelium with a cell wall composed of chitin, cellulose, and other complex polysaccharides. The filamentous fungi are morphologically, physiologically, and genetically distinct from yeasts. Vegetative growth by filamentous fungi is by hyphal elongation and carbon catabolismis obligatory aerobic. The filamentous fungal cell may be a cell of a species of, but not limited to, Trichoderma, (e.g., Trichoderma reesei, the asexual morph of Hypocrea jecorina, previously classified as T. longibrachiatum, Trichoderma viride, Trichoderma koningii, Trichoderma harziamum) (Sheir-Neirs et al, Appl. Microbiol. Biotechnol 20:46-53, 1984; ATCC No. 56765 and ATCC No. 26921); Penicillium sp., Humicola sp. (e.g., H. insolens, H. lanuginose, or H. grisea); Chrysosporium sp. (e.g., C. lucknowense), Gliocladium sp., Aspergillus sp. (e.g., A. oryzae, A. niger, A sojae, A. japonicus, A. nidulans, or A. awamori) (Ward et al., Appl. Microbiol. Biotechnol. 39: 7380743, 1993 and Goedegebuur et al, Genet 41:89-98, 2002), Fusarium sp., (e.g., F. roseum, F. gramimim F. cerealis, F. oxysporuim, or F. venenatum), Neurospora sp., (e.g., N. crassa), Hypocrea sp., Mucor sp., (e.g., M. miehei), Rhizopus sp. and Emericella sp. (see also, Innis et al., Sci. 228:21-26, 1985). The term “Trichoderma” or “Trichoderma sp.” or “Trichoderma spp.” refer to any fungal genus previously or currently classified as Trichoderma.

In some embodiments, the fungus is A. nidulans, A. awamori, A. oryzae, A. aculeatus, A. niger, A. japonicus, T. reesei, T. viride, F. oxysporum, or F. solani. Aspergillus strains are disclosed in Ward et al., Appl. Microbiol. Biotechnol. 39:738-743, 1993 and Goedegebuur et al., Curr Gene 41:89-98, 2002, which are each hereby incorporated by reference in their entireties, particularly with respect to fungi. In particular embodiments, the fungus is a strain of Trichoderma, such as a strain of T. reesei. Strains of T. reesei are known and non-limiting examples include ATCC No. 13631, ATCC No. 26921, ATCC No. 56764, ATCC No. 56765, ATCC No. 56767, and NRRL 15709, which are each hereby incorporated by reference in their entireties, particularly with respect to strains of T. reesei. In some embodiments, the host strain is a derivative of RL-P37. RL-P37 is disclosed in Sheir-Neiss et al., Appl. Microbiol. Biotechnology 20:46-53, 1984, which is hereby incorporated by reference in its entirety, particularly with respect to strains of T. reesei.

In some embodiments, the cell is a yeast, such as Saccharomyces sp., Schizosaccharomyces sp., Pichia sp., or Candida sp.

In some embodiments, the cell is a bacterium, such as strains of Bacillus such as B. lichenformis or B. subtilis, strains of Pantoea such as P. citrea, strains of Pseudomonas such as P. alcaligenes, strains of Streptomyces such as S. lividans or S. rubiginosus, or strains of Escherichia such as E. coli.

As used herein, “the genus Bacillus” includes all species within the genus “Bacillus” as known to those of skill in the art, including but not limited to B. subtilis, B. licheniformis, B. lentus, B. brevis, B. stearothermophilus, B. alkalophilus, B. amyloliquefaciens, B. velezensis, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In some embodiments, the cell is a gram-positive bacterium. Non-limiting examples include strains of Streptomyces (e.g., S. lividans, S. coelicolor, or S. griseus) and Bacillus. In some embodiments, the source organism is a gram-negative bacterium, such as E. coli or Pseudomonas sp.

In some embodiments, the cell is a plant, such as a plant from the family Fabaceae, such as the Faboideae subfamily. In some embodiments, the source organism is kudzu, poplar (such as Populus alba x tremula CAC35696), aspen (such as Populus tremuloides), or Quercus robur.

In some embodiments, the cell is an algae, such as a green algae, red algae, glaucophytes, chlorarachniophytes, euglenids, chromista, or dinoflagellates.

In some embodiments, the cell is a cyanobacteria, such as cyanobacteria classified into any of the following groups based on morphology: Chroococcales, Pleurocapsales, Oscillatoriales, Nostocales, or Stigonematales.

In other embodiments, the cells include strains belonging to the genus Prevotella (such as P. copri), Akkermansia (such as A. municiphilia), Megasphaera (such as M. elsdenii), Oscillibacter, Bacteroides, Bifidobacterium, Brevibacteria, Clostridium, Enterococcus, Escherichia coli, Lactobacillus, Lactococcus, Saccharomyces, and Staphylococcus, Bacteroides fragilis, Bacteroides subtilis, Bacteroides thetaiotaomicron, Bifidobacterium bifidum, Bifidobacterium infantis, Bifidobacterium animalis, Bifidobacterium animalis subsp. lactis 420, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactobacillus crispatus, Lactococcus lactis and, Saccharomyces boulardii.

In some aspects, the cells cultured in liquid media according to the methods disclosed herein are probiotics. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to, certain strains belonging to the genus Bifidobacteria, Escherichia coli, Lactobacillus, and Saccharomyces e.g., Bifidobacterium bifidum, Enterococcus faecium, Escherichia coli strain Nissle, Lactobacillus acidophilus, Lactobacillus bulgaricus, Lactobacillus paracasei, and Lactobacillus plantarum, and Saccharomyces boulardii. The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

In additional embodiments, the cell is a mammalian cell, such as a Chinese hamster ovary (CHO) cell or human embryonic kidney (HEK) cell. The mammalian cell can also be a primary cell, such as a stem cell or an immune cell. Any immune cell can be used such as, without limitation, natural killer (NK) cells, lymphocytes (such as B lymphocytes or T lymphocytes), leukocytes, or monocytes (such as macrophages).

The term “leukocyte” as used herein, refers to cells called white blood cells that help the body fight infections and other diseases, and include for instance granulocytes (e.g., neutrophils, eosinophils, basophils), mononuclear phagocytes, and lymphocytes (e.g., B cells, T cells, natural killer cells).

As used herein, “B cells” or “B lymphocytes” are one of two major classes of lymphocytes. B cells are the precursors of antibody secreting cells, plasma cells, and as such are central to the induction of humoral immune responses. The induction of most humoral immune responses in the adult involves a number of cellular interactions among thymus-derived T lymphocytes, commonly called T cells, antigen presenting cells (APC), and B cells (J. Exp. Med 147:1159, 1978; PNAS 77:1612, 1982; PNAS 79:1989, 1982; Immunol. Rev. 95:914, 1987).

As used herein, “T cells” or “T lymphocytes” are a subset of lymphocytes defined by their development in the thymus and by the presence of heterodimeric receptors associated with proteins of the CD3 complex. T cells can be further divided into helper T cells (CD4+ cells) and cytotoxic T cells (CD8+ cells).

“Natural killer cells” or “NK cells” are a class of large lymphocytes which are an important component of the innate immune system.

The term “monocyte”, as used herein, refers to a type of white blood cells that have two main functions in the immune system: (1) replenish resident macrophages and dendritic cells under normal states, and (2) in response to inflammation signals, monocytes can move quickly (approx. 8-12 hours) to sites of infection in the tissues and divide/differentiate into macrophages and dendritic cells to elicit an immune response. Half of them are stored in the spleen. The term monocyte includes, without limitation both the classical monocyte and the non-classical pro-inflammatory monocyte, which are both present in human blood.

The term “macrophage”, as used herein, refers to CD14+ positive cells derived from the differentiation of the monocytes characterized in being phagocytes, acting in both non-specific defense (innate immunity) as well as to help initiate specific defense mechanisms (adaptive immunity) of vertebrate animals. Their role is to phagocytose (engulf and then digest) cellular debris and pathogens either as stationary or as mobile cells, and to stimulate lymphocytes and other immune cells to respond to the pathogen.

In other embodiments, the animal cell is an organoid. The term “organoid” as used herein refers to an agglomeration of cells that recapitulates aspects of cellular self-organization, architecture and signaling interactions present in a native organ. The term “organoid” includes spheroids or cell clusters formed from suspension cell cultures.

### C. Exemplary Liquid Cell Culture Media

As used herein, the terms “medium” or “media” refer to a liquid growth medium containing the nutrients possible for cell growth. A medium typically contains: (1) a carbon source for cellular growth; (2) various salts, which can vary among species and growing conditions; (3) one or more sources of protein or amino acids; and (4) water. The carbon source can vary significantly, from simple sugars like glucose to more complex hydrolysates of other biomass, such as yeast extract. The salts generally provide essential elements such as magnesium, nitrogen, phosphorus, and sulfur to allow the cells to synthesize proteins and nucleic acids. The medium can also be supplemented with selective agents, such as antibiotics, to select for the maintenance of certain plasmids and the like. For example, if a microorganism is resistant to a certain antibiotic, such as ampicillin or tetracycline, then that antibiotic can be added to the medium in order to prevent cells lacking the resistance from growing. Medium can be supplemented with other compounds as necessary to select for desired physiological or biochemical characteristics, such as particular amino acids and the like.

Any carbon source can be used to cultivate cells. The term “carbon source” refers to one or more carbon-containing compounds capable of being metabolized by a cell or organism. For example, the medium used to cultivate cells can include any carbon source suitable for maintaining the viability or growing the cells.

In some embodiments, the carbon source is a carbohydrate (such as monosaccharide, disaccharide, oligosaccharide, or polysaccharids), invert sugar (e.g., enzymatically treated sucrose syrup), glycerol, glycerine (e.g., a glycerine byproduct of a biodiesel or soap-making process), dihydroxyacetone, one-carbon source, oil (e.g., a plant or vegetable oil such as corn, palm, or soybean oil), animal fat, animal oil, fatty acid (e.g., a saturated fatty acid, unsaturated fatty acid, or polyunsaturated fatty acid), lipid, phospholipid, glycerolipid, monoglyceride, diglyceride, triglyceride, polypeptide (e.g., a microbial or plant protein or peptide), renewable carbon source (e.g., a biomass carbon source such as a hydrolyzed biomass carbon source), yeast extract, component from a yeast extract, polymer, acid, alcohol, aldehyde, ketone, amino acid, succinate, lactate, acetate, ethanol, or any combination of two or more of the foregoing. In some embodiments, the carbon source is a product of photosynthesis, including, but not limited to, glucose.

Exemplary monosaccharides include glucose and fructose; exemplary oligosaccharides include lactose and sucrose, and exemplary polysaccharides include starch and cellulose. Exemplary carbohydrates include C6 sugars (e.g., fructose, mannose, galactose, or glucose) and C5 sugars (e.g., xylose or arabinose). In some embodiments, the cell medium includes a carbohydrate as well as a carbon source other than a carbohydrate (e.g., glycerol, glycerine, dihydroxyacetone, one-carbon source, oil, animal fat, animal oil, fatty acid, lipid, phospholipid, glycerolipid, monoglyceride, diglyceride, triglyceride, renewable carbon source, or a component from a yeast extract). In some embodiments, the cell medium includes a carbohydrate as well as a polypeptide (e.g., a microbial or plant protein or peptide). In some embodiments, the microbial polypeptide is a polypeptide from yeast or bacteria. In some embodiments, the plant polypeptide is a polypeptide from soy, corn, canola, jatropha, palm, peanut, sunflower, coconut, mustard, rapeseed, cottonseed, palm kernel, olive, safflower, sesame, or linseed.

In some embodiments, the concentration of the carbohydrate is at least or about 5 grams per liter of broth (g/L, wherein the volume of broth includes both the volume of the cell medium and the volume of the cells), such as at least or about 10, 15, 20, 30, 40, 50, 60, 80, 100, 150, 200, 300, 400, or more g/L. In some embodiments, the concentration of the carbohydrate is between about 50 and about 400 g/L, such as between about 100 and about 360 g/L, between about 120 and about 360 g/L, or between about 200 and about 300 g/L. In some embodiments, this concentration of carbohydrate includes the total amount of carbohydrate that is added before and/or during the culturing of the host cells.

In some embodiments, the cells are cultured under limited glucose conditions. By “limited glucose conditions” is meant that the amount of glucose that is added is less than or about 105% (such as about 100%) of the amount of glucose that is consumed by the cells. In particular embodiments, the amount of glucose that is added to the culture medium is approximately the same as the amount of glucose that is consumed by the cells during a specific period of time. In some embodiments, the rate of cell growth is controlled by limiting the amount of added glucose such that the cells grow at the rate that can be supported by the amount of glucose in the cell medium. In some embodiments, glucose does not accumulate during the time the cells are cultured. In various embodiments, the cells are cultured under limited glucose conditions for greater than or about 1, 2, 3, 5, 10, 15, 20, 25, 30, 35, 40, 50, 60, or 70 hours. In various embodiments, the cells are cultured under limited glucose conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited glucose conditions may allow more favorable regulation of the cells.

In some embodiments, the cells are cultured in the presence of an excess of glucose. In particular embodiments, the amount of glucose that is added is greater than about 105% (such as about or greater than 110, 120, 150, 175, 200, 250, 300, 400, or 500%) or more of the amount of glucose that is consumed by the cells during a specific period of time. In some embodiments, glucose accumulates during the time the cells are cultured.

Exemplary lipids are any substance containing one or more fatty acids that are C4 and above fatty acids that are saturated, unsaturated, or branched. Exemplary oils are lipids that are liquid at room temperature. In some embodiments, the lipid contains one or more C4 or above fatty acids (e.g., contains one or more saturated, unsaturated, or branched fatty acid with four or more carbons). In some embodiments, the oil is obtained from soy, corn, canola, jatropha, palm, peanut, sunflower, coconut, mustard, rapeseed, cottonseed, palm kernel, olive, safflower, sesame, linseed, oleagineous microbial cells, Chinese tallow, or any combination of two or more of the foregoing.

Exemplary fatty acids include compounds of the formula RCOOH, where “R” is a hydrocarbon. Exemplary unsaturated fatty acids include compounds where “R” includes at least one carbon-carbon double bond. Exemplary unsaturated fatty acids include, but are not limited to, oleic acid, vaccenic acid, linoleic acid, palmitelaidic acid, and arachidonic acid. Exemplary polyunsaturated fatty acids include compounds where “R” includes a plurality of carbon-carbon double bonds. Exemplary saturated fatty acids include compounds where “R” is a saturated aliphatic group. In some embodiments, the carbon source includes one or more C₁₂-C₂₂ fatty acids, such as a Cu saturated fatty acid, a C_{M} saturated fatty acid, a Ci₆ saturated fatty acid, a C₁₈ saturated fatty acid, a C₂₀ saturated fatty acid, or a C₂₂ saturated fatty acid. In an exemplary embodiment, the fatty acid is palmitic acid. In some embodiments, the carbon source is a salt of a fatty acid (e.g., an unsaturated fatty acid), a derivative of a fatty acid (e.g., an unsaturated fatty acid), or a salt of a derivative of fatty acid (e.g., an unsaturated fatty acid). Suitable salts include, but are not limited to, lithium salts, potassium salts, sodium salts, and the like. Di- and triglycerols are fatty acid esters of glycerol.

In some embodiments, the concentration of the lipid, oil, fat, fatty acid, monoglyceride, diglyceride, or triglyceride is at least or about 1 gram per liter of broth (g/L, wherein the volume of broth includes both the volume of the cell medium and the volume of the cells), such as at least or about 5, 10, 15, 20, 30, 40, 50, 60, 80, 100, 150, 200, 300, 400, or more g/L. In some embodiments, the concentration of the lipid, oil, fat, fatty acid, monoglyceride, diglyceride, or triglyceride is between about 10 and about 400 g/L, such as between about 25 and about 300 g/L, between about 60 and about 180 g/L, or between about 75 and about 150 g/L. In some embodiments, the concentration includes the total amount of the lipid, oil, fat, fatty acid, monoglyceride, diglyceride, or triglyceride that is added before and/or during the culturing of the host cells. In some embodiments, the carbon source includes both (i) a lipid, oil, fat, fatty acid, monoglyceride, diglyceride, or triglyceride and (ii) a carbohydrate, such as glucose. In some embodiments, the ratio of the lipid, oil, fat, fatty acid, monoglyceride, diglyceride, or triglyceride to the carbohydrate is about 1:1 on a carbon basis (i.e., one carbon in the lipid, oil, fat, fatty acid, monoglyceride, diglyceride, or triglyceride per carbohydrate carbon). In particular embodiments, the amount of the lipid, oil, fat, fatty acid, monoglyceride, diglyceride, or triglyceride is between about 60 and 180 g/L, and the amount of the carbohydrate is between about 120 and 360 g/L.

Exemplary microbial polypeptide carbon sources include one or more polypeptides from yeast or bacteria. Exemplary plant polypeptide carbon sources include one or more polypeptides from soy, corn, canola, jatropha, palm, peanut, sunflower, coconut, mustard, rapeseed, cottonseed, palm kernel, olive, safflower, sesame, or linseed.

Exemplary renewable carbon sources include cheese whey permeate, cornsteep liquor, sugar beet molasses, barley malt, and components from any of the foregoing. Exemplary renewable carbon sources also include glucose, hexose, pentose and xylose present in biomass, such as corn, switchgrass, sugar cane, cell waste of fermentation processes, and protein by-product from the milling of soy, corn, or wheat. In some embodiments, the biomass carbon source is a lignocellulosic, hemicellulosic, or cellulosic material such as, but are not limited to, a grass, wheat, wheat straw, bagasse, sugar cane bagasse, soft wood pulp, corn, corn cob or husk, corn kernel, fiber from corn kernels, corn stover, switch grass, rice hull product, or a by-product from wet or dry milling of grains (e.g., corn, sorghum, rye, triticate, barley, wheat, and/or distillers grains). Exemplary cellulosic materials include wood, paper and pulp waste, herbaceous plants, and fruit pulp. In some embodiments, the carbon source includes any plant part, such as stems, grains, roots, or tubers. In some embodiments, all or part of any of the following plants are used as a carbon source: corn, wheat, rye, sorghum, triticate, rice, millet, barley, cassava, legumes, such as beans and peas, potatoes, sweet potatoes, bananas, sugarcane, and/or tapioca. In some embodiments, the carbon source is a biomass hydrolysate, such as a biomass hydrolysate that includes both xylose and glucose or that includes both sucrose and glucose.

In some embodiments, the renewable carbon source (such as biomass) is pretreated before it is added to the cell culture medium. In some embodiments, the pretreatment includes enzymatic pretreatment, chemical pretreatment, or a combination of both enzymatic and chemical pretreatment (see, for example, Farzaneh et al, Bioresource Technology 96 (18): 2014-2018, 2005; U.S. Pat. Nos. 6,176,176; 6,106,888; which are each hereby incorporated by reference in their entireties, particularly with respect to the pretreatment of renewable carbon sources). In some embodiments, the renewable carbon source is partially or completely hydrolyzed before it is added to the cell culture medium.

In some embodiments, the concentration of the carbon source (e.g., a renewable carbon source) is equivalent to at least or about 0.1, 0.5, 1, 1.5 2, 3, 4, 5, 10, 15, 20, 30, 40, or 50% glucose (w/v). The equivalent amount of glucose can be determined by using standard HPLC methods with glucose as a reference to measure the amount of glucose generated from the carbon source. In some embodiments, the concentration of the carbon source (e.g., a renewable carbon source) is equivalent to between about 0.1 and about 20% glucose, such as between about 0.1 and about 10% glucose, between about 0.5 and about 10% glucose, between about 1 and about 10% glucose, between about 1 and about 5% glucose, or between about 1 and about 2% glucose.

In some aspects, the carbon source includes yeast extract or one or more components of yeast extract. In some aspects, the concentration of yeast extract is 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. In some aspects, the carbon source includes both yeast extract (or one or more components thereof) and another carbon source, such as glucose.

In some embodiments, cells are cultured in a standard medium containing physiological salts and nutrients (see, e.g., Pourquie, J. et al., Biochemistry and Genetics of Cellulose Degradation, eds. Aubert et al., Academic Press, pp. 71-86, 1988 and Ilmen et al., Appl. Environ. Microbiol. 63:1298-1306, 1997, which are each hereby incorporated by reference in their entireties, particularly with respect to cell medias). Exemplary growth media are common commercially prepared media such as Luria Bertani (LB) broth, Sabouraud Dextrose (SD) broth, or Yeast medium (YM) broth. Other defined or synthetic growth media may also be used, and the appropriate medium for growth of particular host cells are known by someone skilled in the art of microbiology or fermentation science.

In addition to an appropriate carbon source, the cell medium desirably contains suitable minerals, salts, cofactors, buffers, and other components known to those skilled in the art suitable for the growth of the cells.

Any liquid medium formulation can be used to cultivate cells in accordance with the methods disclosed herein. Exemplary liquid medium formulations include, for example, MRS media (particularly for the culture of Lactobacillus species). Each liter of MRS media contains dextrose (20 g), gelatin peptone (10 g), beef extract (8 g), sodium acetate (5 g), yeast extract (4 g), ammonium citrate (2 g), dipotassium phosphate (2 g), polysorbate 80 (1 g), magnesium sulfate (0.2 g), and manganese sulfate (0.05 g).

An additional exemplary liquid medium is Brain Heart Infusion (BHI) media. Each liter of BHI media contains infusion from calf brain (200 g), infusion from beef heart (250 g), proteose peptone (10 g), dextrose (2 g), sodium chloride (5 g), disodium phosphate (2.5 g), and a final pH of 7.4+/−0.2.

A further exemplary liquid medium is tryptic soy broth (TSB) media. Each liter of TSB media contains tryptone (pancreatic digest of casein; 17 g), soytone (peptic digest of soybean; 3 g), glucose (2.5 g), sodium chloride (5 g), dipotassium phosphate (2.5 g), and a final pH of 7.3+/−0.2.

Another exemplary liquid medium is Yeast Casitone Fatty Acids Broth with Carbohydrates (YCFAC Broth), with or without added mucin, which is commercially available through Anaerobe Systems (Morgan Hill, CA).

In some embodiments, the media is a conditioned media. The term “conditioned media” as used herein, refers to media in which cells were already grown for a period of time. This media will have secreted factors, including but not limited to, enzymes (however, in some embodiments the conditioned media will lack or have minimal (e.g. less than 5 nM) one or more enzymes that hydrolyze at least one nucleoside triphosphate), growth factors, cytokines and hormone dissolved in it, which can then be transferred by moving the conditioned media to new cells. One skilled in the art will be familiar with producing conditioned media, and the incubation time in order to condition the media can range from 1-5 days depending on the type of cells growing in the media and their concentration within the media.

### D. Exemplary Cell Culture Conditions

Materials and methods suitable for the maintenance and growth of the recombinant cells in liquid media are described infra, e.g., in the Examples section. Other materials and methods suitable for the maintenance and growth of cell cultures are well known in the art. Exemplary techniques can be found in Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass.

Standard cell culture conditions can be used to culture the cells. In some aspects, cells are grown and maintained at an appropriate temperature, gas mixture, and pH (such as at about 20° C. to about 37° C., at about 6% to about 84% CO₂, and at a pH between about 5 to about 9). In some aspects, cells are grown at 35° C. in an appropriate cell medium. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the cells.

In various embodiments, the cells are grown using any known mode of fermentation, such as batch, fed-batch, or continuous processes. In some embodiments, a batch method of fermentation is used. Classical batch fermentation is a closed system where the composition of the media is set at the beginning of the fermentation and is not subject to artificial alterations during the fermentation. Thus, at the beginning of the fermentation the cell medium is inoculated with the desired host cells and fermentation is permitted to occur adding nothing to the system. Typically, however, “batch” fermentation is batch with respect to the addition of carbon source and attempts are often made at controlling factors such as pH and oxygen concentration. In batch systems, the metabolite and biomass compositions of the system change constantly until the time the fermentation is stopped. Within batch cultures, cells moderate through a static lag phase to a high growth log phase and finally to a stationary phase where growth rate is diminished or halted.

In some embodiments, a variation on the standard batch system is used, such as the Fed-Batch system. Fed-Batch fermentation processes comprise a typical batch system with the exception that the carbon source is added in increments as the fermentation progresses. Fed-Batch systems are useful when catabolite repression is apt to inhibit the metabolism of the cells and where it is desirable to have limited amounts of carbon source in the cell medium. Fed-batch fermentations may be performed with the carbon source (e.g., glucose) in a limited or excess amount. Measurement of the actual carbon source concentration in Fed-Batch systems is difficult and is therefore estimated on the basis of the changes of measurable factors such as pH, dissolved oxygen, and the partial pressure of waste gases such as CO₂. Batch and Fed-Batch fermentations are common and well known in the art and examples may be found in Brock, Biotechnology: A Textbook of Industrial Microbiology, Second Edition (1989) Sinauer Associates, Inc., which is hereby incorporated by reference in its entirety, particularly with respect to cell culture and fermentation conditions.

In some embodiments, continuous fermentation methods are used. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. For example, one method maintains a limiting nutrient such as the carbon source or nitrogen level at a fixed rate and allows all other parameters to moderate. In other systems, a number of factors affecting growth can be altered continuously while the cell concentration (e.g., the concentration measured by media turbidity) is kept constant. Continuous systems strive to maintain steady state growth conditions. Thus, the cell loss due to media being drawn off is balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, Biotechnology: A Textbook of Industrial Microbiology, Second Edition (1989) Sinauer Associates, Inc., which is hereby incorporated by reference in its entirety, particularly with respect to cell culture and fermentation conditions.

In some embodiments, bottles of liquid culture are placed in shakers in order to introduce oxygen to the liquid and maintain the uniformity of the culture. In some embodiments, an incubator is used to control the temperature, humidity, shake speed, and/or other conditions in which a culture is grown. The simplest incubators are insulated boxes with an adjustable heater, typically going up to −65° C. More elaborate incubators can also include the ability to lower the temperature (via refrigeration), or the ability to control humidity or CO₂ levels. Most incubators include a timer; some can also be programmed to cycle through different temperatures, humidity levels, etc. Incubators can vary in size from tabletop to units the size of small rooms.

If desired, a portion or all of the cell medium can be changed to replenish nutrients and/or avoid the build up of potentially harmful metabolic byproducts and dead cells. In the case of suspension cultures, cells can be separated from the media by centrifuging or filtering the suspension culture and then resuspending the cells in fresh media. In the case of adherent cultures, the media can be removed directly by aspiration and replaced. In some embodiments, the cell medium allows at least a portion of the cells to divide for at least or about 5, 10, 20, 40, 50, 60, 65, or more cell divisions in a continuous culture (such as a continuous culture without dilution).

In some aspects, the cells are cultured under limited glucose conditions wherein the amount of glucose that is added is less than or about 105% (such as about 100%, 90%, 80%, 70%, 60%, 50%, 40%, 30%, 20%, or 10%) of the amount of glucose that is consumed by the cells. In particular aspects, the amount of glucose that is added to the culture medium is approximately the same as the amount of glucose that is consumed by the cells during a specific period of time. In some aspects, the rate of cell growth is controlled by limiting the amount of added glucose such that the cells grow at the rate that can be supported by the amount of glucose in the cell medium. In some aspects, glucose does not accumulate during the time the cells are cultured.

In various aspects, the cells are cultured in the presence of one or more enzymes that hydrolyze at least one nucleoside triphosphate for greater than or about 1, 2, 3, 5, 10, 15, 20, 25, 30, 35, 40, 50, 60, or 70 hours. In various aspects, the cells are cultured in the presence of one or more enzymes that hydrolyze at least one nucleoside triphosphate under limited glucose conditions for greater than or about 1, 2, 3, 5, 10, 15, 20, 25, 30, 35, 40, 50, 60, or 70 hours. In various aspects, the cells are cultured in the presence of one or more enzymes that hydrolyze at least one nucleoside triphosphate under limited glucose conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited glucose conditions can allow more favorable regulation of the cells.

In some aspects, the cells are grown in batch culture. The cells can also be grown in fed-batch culture or in continuous culture. Additionally, the cells can be cultured in minimal medium. The minimal medium can be further supplemented with 1.0% (w/v) glucose, or any other six carbon sugar, or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract.

In some aspects, the cells can be grown under low oxygen conditions or anaerobic conditions. In other aspects, the cells are grown under atmospheric conditions comprising any of about 4%, 5%, 6%, 7%, 8%, 9%, 10%, 11%, 12%, 13%, 14%, or 15%, inclusive, including any values in between these percentages, oxygen. In other aspects, the cells are grown under atmospheric conditions comprising any of about 3-8%, 3.5-8.5%, 4-9%, 4.5-9.5%, 5-10%, 5.5-10.5%, 6-11%, or 6.5-11.5% oxygen.

### E. Methods for Improving Growth of Cells Cultured in a Liquid Media

Provided herein are methods for improving growth of cells cultured in a liquid media. The cells (for example, probiotic cells or recombinant cells) are cultured in a liquid media (such as any of the liquid media disclosed herein) comprising one or more enzymes that hydrolyze at least one nucleoside triphosphate under any of the conditions disclosed herein. When cultured in this manner, the cells will exhibit improved growth compared to cells (for example, probiotic cells) cultured in media that does not comprise one or more enzymes that hydrolyze at least one nucleoside triphosphate.

In some embodiments, improved growth can be determined by measuring optical density (OD) at a 600 nm wavelength (OD₆₀₀). OD₆₀₀ is a commonly used spectrophotometric method for estimating the concentration of bacteria or other cells in a liquid. Measuring the concentration can indicate the growth stage of cultured cell population, i.e. whether it is in the lag phase, log phase, or stationary phase. This is done by measuring the absorbance of the OD₆₀₀ light with the use of a spectrophotometer. A growth curve can then be constructed by taking absorbance measurements as a function of time. In some embodiments, cells (including probiotic cells or recombinant cells) exhibit at least about a 10%-500% increase in OD₆₀₀, such as any of about 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 150%, 160%, 170%, 180%, 190%, 200%, 210%, 220%, 230%, 240%, 250%, 260%, 270%, 280%, 290%, 300%, 325%, 350%, 375%, 400%, 425%, 450%, 475%, or 500% or more increase in OD₆₀₀, including all values falling in between these percentages, when cultured in liquid media comprising one or more enzymes that hydrolyze at least one nucleoside triphosphate compared to cells (including probiotic cells or recombinant cells) cultured in media that does not comprise one or more enzymes that hydrolyze at least one nucleoside triphosphate.

In other embodiments, improved growth can be determined by measuring colony forming units (cfu)/mL. A colony-forming unit (CFU, cfu, Cfu) is a unit used in microbiology to estimate the number of viable cells in a sample. Viable is defined as the ability to multiply via binary fission under the controlled conditions. Counting with colony-forming units requires culturing the microbes and counting only viable cells, in contrast with microscopic examination which counts all cells, living or dead. The visual appearance of a colony in a cell culture requires significant growth, and when counting colonies it is uncertain if the colony arose from one cell or a group of cells. Expressing results as colony-forming units reflects this uncertainty. Cfu's can be determined manually by visual inspection or can be enumerated from pictures of plates using software tools. In some embodiments, cells (including probiotic cells or recombinant cells) exhibit at least about a 10%-500% increase in Cfu/mL, such as any of about 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 150%, 160%, 170%, 180%, 190%, 200%, 210%, 220%, 230%, 240%, 250%, 260%, 270%, 280%, 290%, 300%, 325%, 350%, 375%, 400%, 425%, 450%, 475%, or 500% or more increase in Cfu/mL, including all values falling in between these percentages, when cultured in liquid media comprising one or more enzymes that hydrolyze at least one nucleoside triphosphate compared to cells (including probiotic cells or recombinant cells) cultured in media that does not comprise one or more enzymes that hydrolyze at least one nucleoside triphosphate.

In other embodiments, improved growth can be determined by measuring cell lysis or post-culture viability. Numerous techniques are known in the art for assessing the health and viability of cells post culture such as, for example, the dye exclusion method using live membrane impermeable dyes such as trypan blue. In some embodiments, cells (including probiotic cells or recombinant cells) exhibit at least about a 10%-500% decrease in lysis, such as any of about 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 150%, 160%, 170%, 180%, 190%, 200%, 210%, 220%, 230%, 240%, 250%, 260%, 270%, 280%, 290%, 300%, 325%, 350%, 375%, 400%, 425%, 450%, 475%, or 500% or more decrease in lysis, including all values falling in between these percentages, when cultured in liquid media comprising one or more enzymes that hydrolyze at least one nucleoside triphosphate compared to cells (including probiotic cells or recombinant cells) cultured in media that does not comprise one or more enzymes that hydrolyze at least one nucleoside triphosphate. In other embodiments, cells (including probiotic cells or recombinant cells) exhibit at least about a 10%-500% increase in post-culture viability, such as any of about 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 150%, 160%, 170%, 180%, 190%, 200%, 210%, 220%, 230%, 240%, 250%, 260%, 270%, 280%, 290%, 300%, 325%, 350%, 375%, 400%, 425%, 450%, 475%, or 500% or more increase in post-culture viability, including all values falling in between these percentages, when cultured in liquid media comprising one or more enzymes that hydrolyze at least one nucleoside triphosphate compared to cells (including probiotic cells or recombinant cells) cultured in media that does not comprise one or more enzymes that hydrolyze at least one nucleoside triphosphate.

In yet other embodiments, improved growth can be determined by measuring proliferation rate. The term “proliferation rate,” as used herein, refers to the change in the number of cells per unit time or the change in the number of cells exhibiting a marker of progression through the cell cycle toward cell division, per unit time. Such markers may be, without limitation, morphological, indicators of DNA replication or expressed gene products. In some embodiments, cells (including probiotic cells or recombinant cells) exhibit at least about a 10%-500% increase in proliferation rate, such as any of about 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 150%, 160%, 170%, 180%, 190%, 200%, 210%, 220%, 230%, 240%, 250%, 260%, 270%, 280%, 290%, 300%, 325%, 350%, 375%, 400%, 425%, 450%, 475%, or 500% or more increase in proliferation rate, including all values falling in between these percentages, when cultured in liquid media comprising one or more enzymes that hydrolyze at least one nucleoside triphosphate compared to cells (including probiotic cells or recombinant cells) cultured in media that does not comprise one or more enzymes that hydrolyze at least one nucleoside triphosphate.

In another embodiment, improved growth can be determined by measuring increased cell mass. As used herein, “cell mass,” “cell density,” and the like refer to a collection of cells. For example, a cell mass can refer to a cell pellet or the overall number or weight of cells that comprise the cell pellet. As used herein, “unit of cell mass” and the like reflects a number of ways of representing cell mass, e.g., cell number, cell density, cell volume, packed cell volume, dry cell weight, etc. A person skilled in the art will readily comprehend that, depending on the unit of cell mass, the peak units of cell mass will be represented by either peak cell numbers, peak cell density, peak cell volume, peak packed cell volume, peak dry weight, etc. In some embodiments, cells (including probiotic cells or recombinant cells) exhibit at least about a 10%-500% increased cell mass, such as any of about 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 150%, 160%, 170%, 180%, 190%, 200%, 210%, 220%, 230%, 240%, 250%, 260%, 270%, 280%, 290%, 300%, 325%, 350%, 375%, 400%, 425%, 450%, 475%, or 500% or more increased cell mass, including all values falling in between these percentages, when cultured in liquid media comprising one or more enzymes that hydrolyze at least one nucleoside triphosphate compared to cells (including probiotic cells or recombinant cells) cultured in media that does not comprise one or more enzymes that hydrolyze at least one nucleoside triphosphate.

In still another embodiment, improved growth can be determined by measuring increased cell numbers. As used herein, “cell number” refers to the absolute number of cells in a given culture. Cell number can be assessed using any number of means known in the art such as, without limitation, by counting with a Coulter counter or by use of a hemocytometer. In some embodiments, cells (including probiotic cells or recombinant cells) exhibit at least about a 10%-500% increased cell numbers, such as any of about 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 150%, 160%, 170%, 180%, 190%, 200%, 210%, 220%, 230%, 240%, 250%, 260%, 270%, 280%, 290%, 300%, 325%, 350%, 375%, 400%, 425%, 450%, 475%, or 500% or more increased cell numbers, including all values falling in between these percentages, when cultured in liquid media comprising one or more enzymes that hydrolyze at least one nucleoside triphosphate compared to cells (including probiotic cells or recombinant cells) cultured in media that does not comprise one or more enzymes that hydrolyze at least one nucleoside triphosphate.

### F. Methods for Producing Bacteria for use in Production of Fermented Dairy Products

Also provided herein is a method for producing bacteria for use in production of fermented dairy products, the method comprising culturing the bacteria in a liquid media comprising one or more enzymes that hydrolyze at least one nucleoside triphosphate, wherein the bacteria exhibit improved growth compared to bacteria cultured in media that does not comprise one or more enzymes that hydrolyze at least one nucleoside triphosphate.

The bacteria so produced may be used to inoculate a milk substrate to facilitate thermophilic fermentation. A “milk substrate” for producing the thermophilic fermented milk may be any raw and/or processed milk material that can be subjected to fermentation, e.g., thermophilic fermentation, according to the methods provided herein. Thus, useful milk substrates include, but are not limited to, solutions/suspensions of any milk or milk-like products comprising protein, such as full fat or reduced fat milk, skim milk, buttermilk, reconstituted milk powder, condensed milk, dried milk, whey, whey permeate, whey protein concentrate, or cream. In some embodiments, the milk substrate may originate from any mammal, e.g., being substantially pure mammalian milk, or reconstituted milk powder.

In some embodiments, the one or more bacteria produced in accordance with the methods disclosed herein are contained in bacterial compositions, such as a starter culture or non-starter culture. In some embodiments, the one or more bacterial strains are contained in a starter culture. In some embodiments, the starter culture is a preparation of living bacteria able to assist in fermentation, e.g., thermophilic fermentation. In some embodiments, the milk substrate is inoculated with a starter culture. In some embodiments, the one or more bacterial strains are contained in a non-starter culture, such as a protective culture. In some embodiments, a protective culture is a culture able to reduce or prevent the growth of biological contaminants, such as yeast and mold. In some embodiments, the milk substrate is inoculated with a starter culture and a non-starter culture, e.g., a protective culture. The terms inoculating and adding may be used interchangeably to refer to contacting a milk substrate with with one or more bacteria, for example as contained in a bacterial composition, e.g., starter culture, protective culture.

In some embodiments, the milk substrate is inoculated with one or more bacterial strains separately. For example, the strains are not mixed together prior to being added to the milk substrate. In some embodiments, the strains are mixed together prior to being added to the milk substrate. Regardless of how a strain is added to the milk substrate, the strain or mixtures of strains used for inoculation may be referred to as or included in a starter culture or protective culture.

In some embodiments, thermophilic microorganisms, such as thermophilic bacteria, refer to microorganisms that function preferentially at temperatures above 43° C. The industrially most useful thermophilic bacteria include Streptococcus spp. and Lactobacillus spp.

In some embodiments, the starter culture is a pure culture, i.e., comprises or consists of a single bacterial strain. In some embodiments, the starter culture is a mixed culture, i.e. comprises or consists of at least one bacterial strain of the invention as described herein and at least one other bacterial strain. For example, at least 1 or more, and in particular 1, 2, 3, 4 or 5, other bacterial strains are included in the starter culture.

In some embodiments, the starter culture contains one or more lactic acid bacteria. As it is normal in lactic acid bacterial fermentation processes to apply a mixed culture as a starter culture, the composition may in some embodiments include a multiplicity of strains either belonging to the same species or belonging to different species. For example, in some cases, the lactic acid bacteria in the starter culture is or includes a mixture of a Lactobacillus dulbrueckii subsp bulgaricus strain and a Streptococcus thermophilus strain.

In some embodiments, the starter culture includes at least bacteria of the genera Streptococcus and Lacticaseibacillus. In some embodiments, the starter culture includes or consists of the genera Lactococcus, Lactobacillus, Streptococcus, Lacticaseibacillus, Leuconostoc, Pediococcus, Enterococcus, Bifidobacterium, Paralactobacillus, Acetilactobacillus, Agrilactobacillus, Amylolactobacillus, Apilactobacillus, Bombilactobacillus, Companilactobacillus, Dellaglioa, Fructilactobacillus, Furfurilactobacillus, Holzapfelia, Lacticaseibacillus, Lactiplantibacillus, Lapidilactobacillus, Latilactobacillus, Lentilactobacillus, Levilactobacillus, Ligilactobacillus, Limosilactobacillus, Liquorilactobacillus, Loigolactobacilus, Paucilactobacillus, Schleiferilactobacillus, Secundilactobacillus, or any combination thereof. In some embodiments, the starter culture includes or consists of the genera Lactococcus, Lactobacillus, Streptococcus, Lacticaseibacillus, Leuconostoc, Pediococcus, Enterococcus or Bifidobacterium, or any combination thereof. In some embodiments, the starter culture includes or consists of the genera Lactococcus, Lactobacillus, Streptococcus, Lacticaseibacillus, Leuconostoc, Pediococcus, or Bifidobacterium, or any combination thereof.

In some embodiments, the starter culture includes one or more of a Streptococcus thermophilus strain, a Lactobacillus acidophilus strain, a Lacticaseibacillus rhamnosus strain, a Bifidobacterium lactis strain, a Limosilactobacillus fermentum strain, a Lacticaseibacillus paracasei strain, a Lactiplantibacillus plantarum strain, a Lactobacillus dulbrueckii subsp bulgaricus strain, a Propionibacteria freudenreichii strain, a Pediococcus acidilactici strain, an Enterococcus faecium strain, and a Lactococcus lactis strain, or any combination of the foregoing. In some embodiments, the starter culture includes one or more of a Streptococcus thermophilus strain, a Lactobacillus acidophilus strain, a Lacticaseibacillus rhamnosus strain, a Bifidobacterium lactis strain, a Limosilactobacillus fermentum strain, a Lacticaseibacillus paracasei strain, a Lactiplantibacillus plantarum strain, a Lactobacillus dulbrueckii subsp bulgaricus strain, a Propionibacteria freudenreichii strain, a Pediococcus acidilactici strain, and a Lactococcus lactis strain, or any combination of the foregoing.

In some embodiments, the starter culture includes at least one Lactococcus lactis strain. For example, the starter culture may include any Lactococcus lactis strain known in the art, such as strains from the Lactococcus lactis subsp. cremoris, Lactococcus lactis subsp. hordniae or Lactococcus lactis subsp. lactis. In some embodiments, the starter culture comprises a Lactococcus lactis subsp. cremoris strain and/or a Lactococcus lactis subsp. lactis strain.

In some embodiments, the starter culture includes one or more Lacticaseibacillus rhamnosus strains. In some embodiments, the starter culture includes Lacticaseibacillus rhamnosus strain DGCC1179 that was deposited with the German Collection of Microorganisms and Cell Cultures (DSMZ) under accession no. DSM 33650 or a mutant strain thereof, wherein the mutant strain is obtained by using the deposited strain as starting material. In some embodiments, the mutant strain is a strain having all of the identifying characteristics of the strain deposited at DSM under number DSM 336520.

In some embodiments, the starter culture includes one or more Streptococcus thermophilus strains. In some embodiments, the starter culture includes Streptococcus thermophilus strain DGCC11042 that was deposited with the German Collection of Microorganisms and Cell Cultures (DSMZ) under accession no. DSM 336521 or a mutant strain thereof, wherein the mutant strain is obtained by using the deposited strain as starting material. In some embodiments, the mutant strain is a strain having all of the identifying characteristics of the strain deposited at DSM under number DSM 336521.

In some embodiments, the starter culture includes one or more strains of Lacticaseibacillus rhamnosus and one or more strains of Streptococcus thermophilus.

The invention can be further understood by reference to the following examples, which are provided by way of illustration and are not meant to be limiting.

### G. Methods for Improving the Growth of Cells Engineered to Produce Recombinant Protein

Also provided herein is a method for improving the growth of cells engineered to produce a recombinant protein. The engineered cells are cultured in a media containing one or more enzymes that hydrolyze at least one nucleoside triphosphate.

The cells can exhibit improved growth compared to cells cultured in media that does not comprise one or more enzymes that hydrolyze at least one nucleoside triphosphate. In some embodiments, the one or more enzymes that hydrolyze at least one nucleoside triphosphate (such as any of the enzymes that hydrolyze at least one nucleoside triphosphate disclosed herein) is exogenously added to the media and/or recombinantly expressed by the engineered cells in addition to the first recombinant protein (i.e. the same recombinant cell can recombinantly express both a first recombinant protein of interest and one or more enzymes that hydrolyze at least one nucleoside triphosphate simultaneously such as, for example, one or more of enzymes encoded by SEQ ID NOs: 1, 2, 3, 4, 5, and/or 6 or at least about 60% identical to (such as any of about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to one or more of the enzymes encoded by SEQ ID NOs: 1, 2, 3, 4, 5, and/or 6)).

Any of the cells disclosed herein can be engineered to produce a recombinant protein and cultured in accordance with the methods disclosed herein. Thus, in certain embodiments, provided herein are cells expressing recombinant proteins, recombinant polynucleotides (e.g. vectors, expression cassettes) encoding one or more heterologous proteins particularly suitable for introducing (e.g., transforming) into host cells (i.e., for the expression of recombinant proteins) and the like.

Thus, certain embodiments are related to, inter alia, nucleic acids, polynucleotides (e.g., plasmids, vectors, expression cassettes), regulatory elements, and the like, suitable for use in constructing recombinant host cells for culturing in the presence of one or more enzymes that hydrolyze at least one nucleoside triphosphate. Accordingly, as generally described herein, recombinant cells of the disclosure may be constructed by one of skill using standard and routine recombinant DNA and molecular cloning techniques well known in the art. Methods for genetically modifying cells include, but are not limited to, (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) a gene down-regulation, (f) site specific mutagenesis and/or (g) random mutagenesis.

Those of skill in the art are well aware of suitable methods for introducing polynucleotide sequences into host cells. Indeed, such methods as transformation including protoplast transformation and congression, transduction, and protoplast fusion are known and suited for use in the present disclosure. Methods of transformation are particularly suitable to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the cell by methods known in the art. In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in Gram-positive cells are generally known on one of skill in the art. Promoter sequences of the disclosure are generally chosen so that they are functional in the host cells.

In some embodiments, improved growth comprises one or more of greater optical density (OD), higher colony forming units (cfu)/mL, decreased cell lysis, higher proliferation rate, increased cell mass, or higher cell numbers as discussed herein. In other embodiments, improved growth comprises higher recombinant protein production.

Accordingly, in some embodiments, improved growth can be determined by measuring recombinant protein production. In some embodiments, recombinant cells exhibit at least about a 10%-500% increase in recombinant protein production, such as any of about 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 150%, 160%, 170%, 180%, 190%, 200%, 210%, 220%, 230%, 240%, 250%, 260%, 270%, 280%, 290%, 300%, 325%, 350%, 375%, 400%, 425%, 450%, 475%, or 500% or more increase in recombinant protein production, including all values falling in between these percentages, when cultured in liquid media comprising one or more enzymes that hydrolyze at least one nucleoside triphosphate compared to recombinant cells cultured in media that does not comprise one or more enzymes that hydrolyze at least one nucleoside triphosphate.

### EXAMPLES

### Example 1: Growth Enhancement of Bifidobacterium animalis Cultured in the Presence of a Phosphatase

This Example shows improved growth of B. animalis when cultured in the presence of an apyrase.

MRS media was inoculated with 1% overnight grown culture of Bifidobacterium animalis. Cells were grown with the addition of 5 mM ATP (Sigma) or 100 nM potato apyrase (ATPase; Sigma catalog #A6535). Experiments were performed in volumes of 200 μl per well in standard microtiter plates. Growth was assessed via Synergy HTX multi-mode plate reader (Bio Tek Instruments) at OD₆₀₀ every 30 minutes for 24 hrs. Each plate was incubated at 37° C. and shaken once every 30 mins just before the reading.

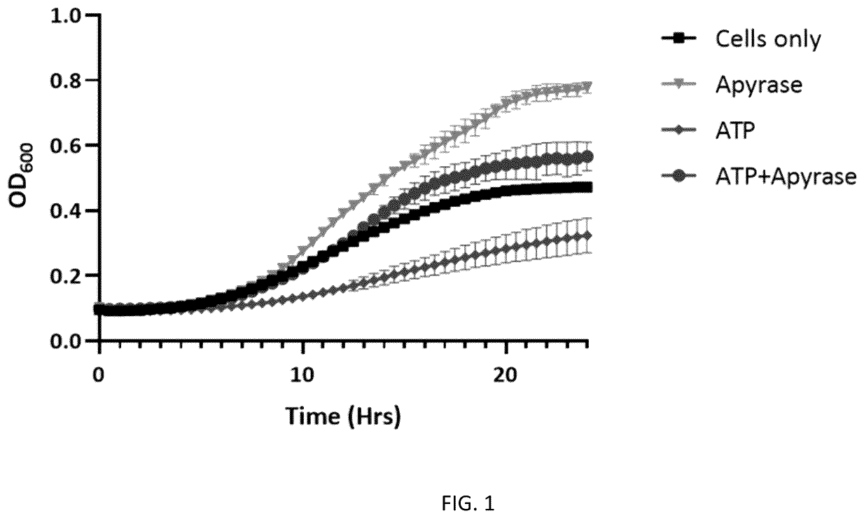

As shown in FIG. 1, cellular growth was supressed in the presence of exogenously added ATP. However, the presence of exogenously-added apyrase negated the growth inhibitory effect of exogenously-added ATP. In the presence of exogenously-added apyrase alone, the cells grew better than the cells-only control. Without being bound to theory, it is believe that the observed improved growth resulted from enzymatic removal of ATP produced and secreated by the cultured bacteria.

### Example 2: Extracellular ATP (eATP) Production by Bifidobacterium animalis Cultured in the Presence of a Phosphatase

This Example assessed the amount of eATP produced by cultured cells in the presence and absence of a phosphatase.

MRS media was inoculated with 1% overnight grown culture of B. animalis subsp. lactis 420. Cells were grown at 37° C. under anaerobic conditions with and without potato apyrase. Potato apyrase was used at 100 nM. 200 μl of the sample was collected at different time points. Cells were harvested by centrifugation at 5000 rpm for 5 mins. Supernatants were used to estimate nanomolar ATP using a Luciferase® Reporter Assay System (Promega).

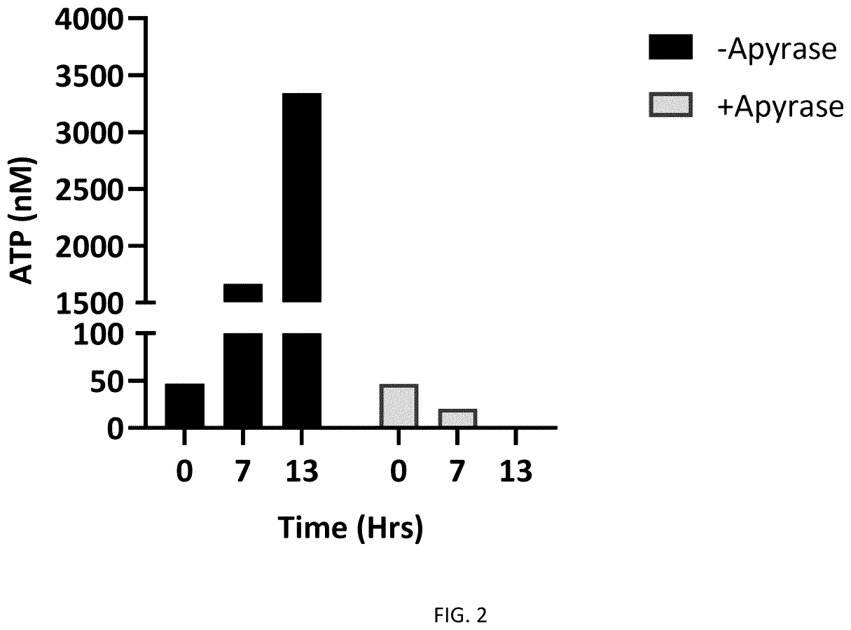

ATP measurements were performed on supernatants collected at 0, 7 and 13 hr time points. As shown in FIG. 2, samples from cultures without exogenously-added apyrase showed accumulation of ATP as growth progressed. In contrast, cultures with exogenously-added apyrase did not exhibit ATP accumulation. Rather, these cultures exhibited a decrease in nanomolar ATP over time (FIG. 2). Without being bound to theory, this data suggested that the growth promoting effect of apyrase observed in Example 1 was due to removal of eATP from the culture.

### Example 3: Growth Enhancement of Bifidobacterium animalis Cultured in the Presence of a Heat-Killed Phosphatase

This Example shows improved growth of B. animalis when cultured in the presence of an apyrase that has been inactivated due to exposure to high temperatures.

MRS media was inoculated with 1% over night grown culture of B. animalis. Cells were grown with 100 nM potato apyrase (ATPase) or heat-treated (95° C. for 10 mins) 100 nM potato apyrase. Experiments were performed in volumes of 200 μl per well in standard microtiter plates. Growth was assessed via plate reader at OD₆₀₀ every 30 minutes.

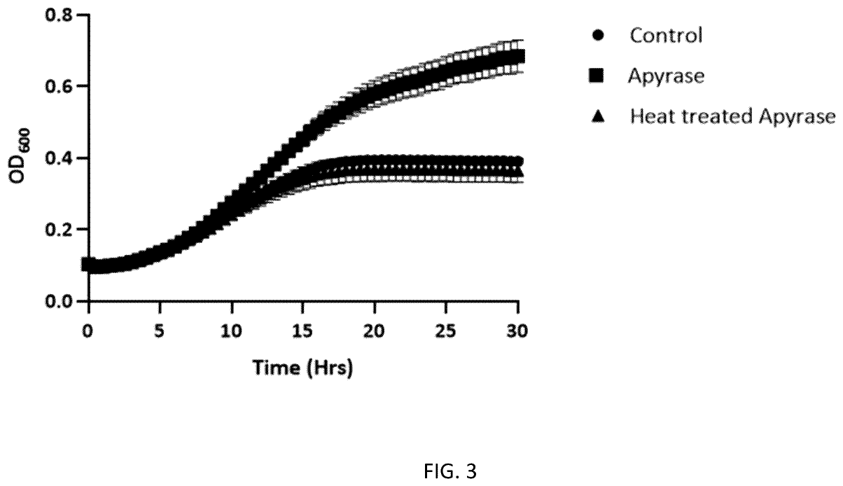

As shown in FIG. 3, cells cultured in the presence of heat-treated apyrase failed to exhibit the growth enhancement shown in FIG. 1, suggesting that the phosphorylase activity of the apyrase is necessary for enhancement of cellular growth.

### Example 4: Growth Enhancement of Multiple Bacterial Species and Yeast Species Cultured in the Presence of a Phosphatase in Various Liquid Media

Different growth media were inoculated with 1% over night grown culture of various bacterial and yeast species (as detailed in Table 1). Cells were grown in the presence of 5 mM exogenously-added ATP or 100 nM potato apyrase or bovine alkaline phosphatase (ATPase). Experiments were done in volumes of 200 μl per well in standard microtiter plates. Growth was assessed via plate reader at OD₆₀₀ every 30 minutes for 48 hours. All the experiments were done at 37° C. anaerobically except for yeast strain Kazachstania unispora where cells were grown aerobically at 28° C.

As shown in Table 1, multiple bacterial strains were sensitive to the presence of eATP and show growth inhibition in its presence. Addition of ATPase was observed to enhance bacterial growth resulting from, without being bound to theory, removal of eATP contributed by bacterial cells or the culture media.

TABLE 1
Effect of ATP and ATPase on bacterial growth

| | Growth | Sensitivity | Improvement in |
|---|---|---|---|
| Strain | media | to ATP | growth by ATPase |
| Bifidobacterium animalis | MRS | YES | +++ (AP) |
| Bifidobacterium lactis | MRS | YES | ++ (apyrase) |
| Bifidobacterium Infantis | MRS | YES | + (apyrase) |
| Bifidobacterium longum | BHI | YES | ++ (apyrase) |
| Lactobacillus rhamnosus | BHI | YES | ++ (apyrase) |
| Prevotella copri | TSA | YES | + (AP) |
| Oscillibacter sp | YCFAC | YES | + (apyrase) |
| Akkermansia sp | YCFAC + | YES | + (apyrase) |
| | Mucin | | |
| Lactobacillus rhamnosus | MRS | YES | ++ (apyrase) |
| Bacillus amyloliquefaciens | TSA | YES | + (apyrase) |
| Lactobacillus crispatus | MRS | YES | ++ (apyrase) |
| Kazachstania unispora | YM | YES | ++ (apyrase) |

AP = alkaline phosphatase;
apyrase = potato apyrase

In conclusion, prokaryotic or eukaryotic cell growth in liquid culture are sensitive to the presence of eATP and this sensitivity is relieved by the presence of an ATPase. When cells perceive ATP in their outer environment, growth is suppressed relative to environments that lack or have reduced eATP. Removal of eATP results in enhancement of cell growth.

### Example 5. Growth Enhancement of CHO Mammalian Cells Cultured in the Presence of Exogenously Added ATPase

The growth effect of exogenously added ATPase enzymes was examined on established mammalian (Chinese hamster ovary (CHO)) cell lines. CHO wild-type cell line was purchased from Creative Biogene (NY, USA) Cat No. CSC-C3064, and CHO cell line constitutively expressing Green Fluorescent Protein (CHO-GFP) was purchased from GenTarget, Inc (San Diego, CA, USA) Cat No. SC039-Puro. Wild type CHO cell line was maintained in CD OptiCHO™ (ThermoFisher) medium for growth in suspension. GFP expressing CHO cell line was maintained in RPMI 1640 media (ThermoFisher) for adherent growth. Cell lines were cultured in a humidified incubation chamber at 37° C. with 5% CO₂.

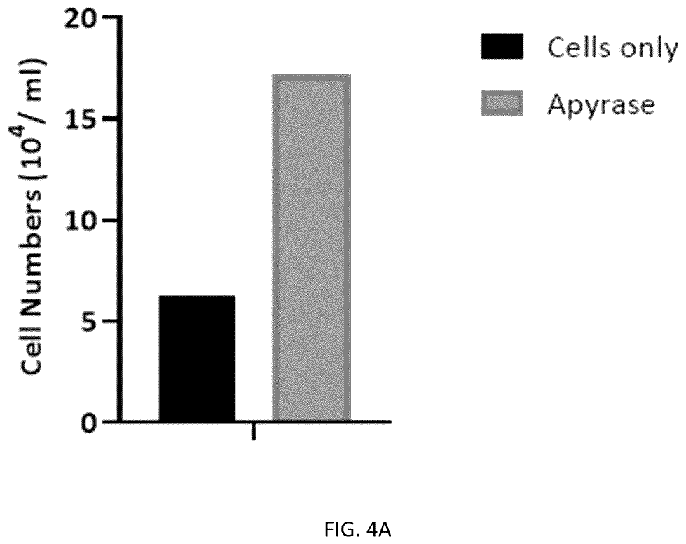

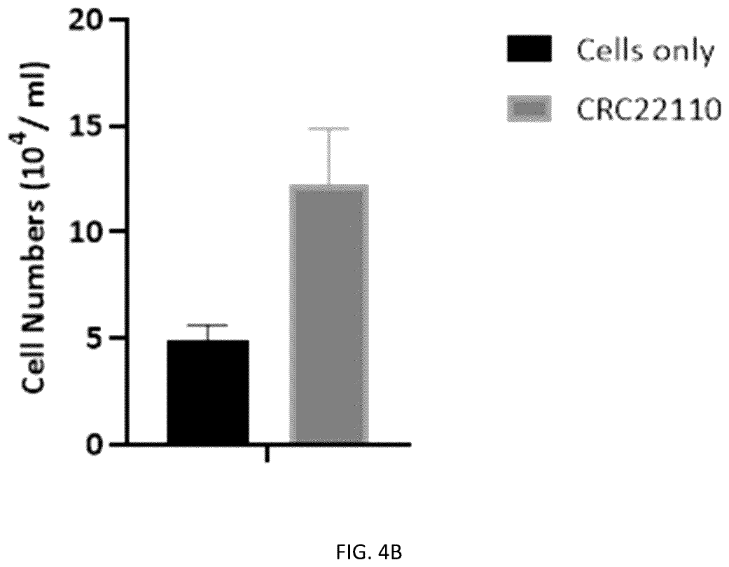

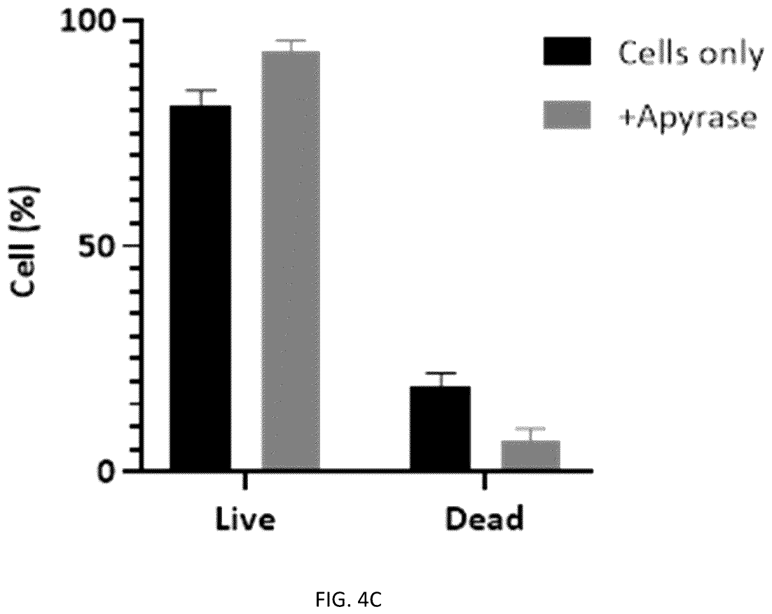

To measure the effect on CHO cells in suspension, cultures were seeded onto the wells of 6-well plates at 1×10⁴ cells per well and incubated for 12 hours. Subsequently, ATPase (potato apyrase or bacterial enzyme CRC22110 (SEQ ID NO:1)) were added to the wells at 10 nM final concentration and incubated for an additional 48 hours. The potato apyrase (Cat No. A6410) and, was sourced from Sigma. The recombinant CRC22110 (SEQ ID NO: 1) enzyme was purified from Bacillus cultures using chromatography methods known in the art. After a total of 60 hours in culture, cells were detached by trypsin treatment, stained with trypan blue and counted in a Countess 3 FL system (Invitrogen). Data on FIG. 4 show the effect of ATPase on CHO cell growth. Panel 4A shows effect of added potato apyrase on total cell count. Panel 4B shows effect of added CRC22110 phosphatase on total cell count. Panel 4C shows the effect of added potato apyrase on the viability of cells using trypan blue exclusion dye method. Results in FIGS. 4A and 4B show that addition of potato apyrase or CRC22110 enzyme leads to a 3-fold increase in CHO cell numbers after 60 hrs of growth in suspension, thus addition of ATPase to the culture media supports growth of CHO cells in suspension. Results in FIG. 4C show that apyrase also improves the number of viable cells in culture.

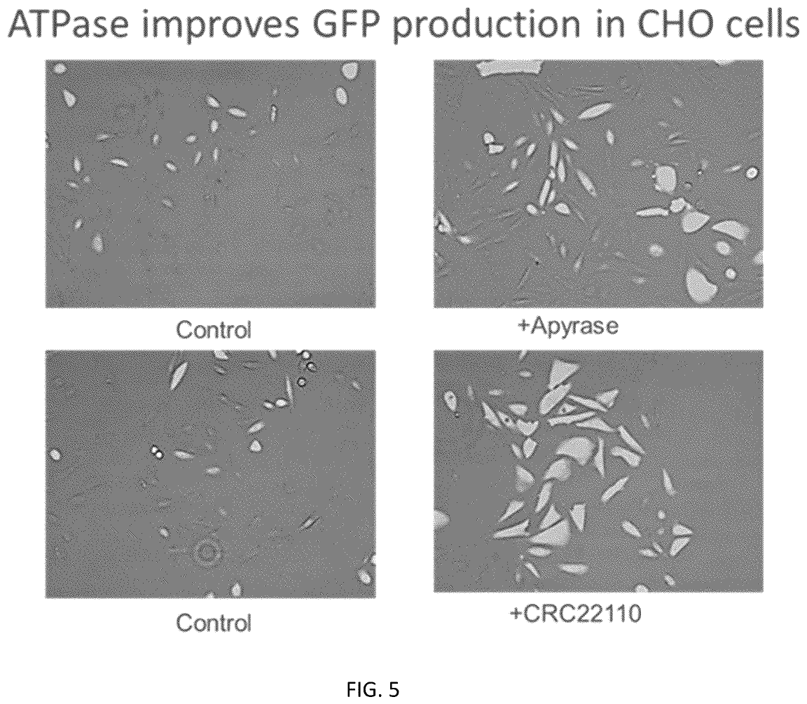

To study the effect of exogenous ATPase on recombinant protein expression by mammalian cells, the CHO-GFP cell line was used. CHO-GFP cells were seeded onto the wells of 6-well plates at 1×10⁴ cells per well and incubated for 12 hours. Subsequently, ATPase (potato Apyrase or bacterial enzyme CRC22110) were added to the wells at 10 nM final concentration and cells were incubated for an additional 48 hours For GFP expression visualization, cells were imaged after 60 hrs of incubation with ATPase by using Invitrogen™ EVOS™ FL Imaging System. FIG. 5 shows fields of CHO-GFP cells grown on the presence or absence of ATPase captured under fluorescence to detect the expression of GFP. The number of cells displaying fluorescence signal and the amount of fluorescence are both increased in the cultures grown with exogenous ATPases, potato apyrase or CRC22110. These images suggest that addition of ATPase trigger increased production of recombinant GFP in the CHO cells.

### SEQUENCES

Predicted mature polypeptide sequence of CRC22110 (Gallaecimonas xiamenensis 3-C-1):

(SEQ ID NO: 1)
ADKPACRAVFDAGSSGTRLFLYVQQEGGWQPWPGAKLGALADPVRQHQGP
KAMAALAADLANSLEALRQDGPTNSEGQPRWQGFDWQQACRLQSVSVLAT
AGMRLAEQQDPVASVALWQEVQQALAGLLGPAVAIDARTLTGFEEGFYAW
LAARARLGRSDMGIVEMGGASSQVAFPCPACDRADDAVQQVRLEGQTLDF
YSYSFLGLGQDEAPKVLGVPHACRYGAGVKDPNWQLGQCQAQLPLTLGRA
LKDPYNFQGQGQGGYRQVPTEKAGVKDWLLTGAFQYNTPDQLDSCCLNQG
QCYQPSTACFRVAYLPLYLDSLGVDPHSPDLDVSWTLGAVLCRNEQCLAP
SKPPCRWRKAGCLAPSVGDGRPEGRPGQQPGGVQHRQH

Predicted mature polypeptide sequence of CRC22110 (Gallaecimonas xiamenensis 3-C-1):

(SEQ ID NO: 2)
AGKADKPACRAVEDAGSSGTRLFLYVQQEGGWQPWPGAKLGALADPVRQH
QGPKAMAALAADLANSLEALRQDGPTNSEGQPRWQGFDWQQACRLQSVSV
LATAGMRLAEQQDPVASVALWQEVQQALAGLLGPAVAIDARTLTGFEEGF
YAWLAARARLGRSDMGIVEMGGASSQVAFPCPACDRADDAVQQVRLEGQT
LDFYSYSFLGLGQDEAPKVLGVPHACRYGAGVKDPNWQLGQCQAQLPLTL
GRALKDPYNFQGQGQGGYRQVPTEKAGVKDWLLTGAFQYNTPDQLDSCCL
NQGQCYQPSTACFRVAYLPLYLDSLGVDPHSPDLDVSWTLGAVLCRNEQC
LAPSKPPCRWRKAGCLAPSVGDGRPEGRPGQQPGGVQHRQH

Sequence of potato apyrase (bold=signal sequence):

(SEQ ID NO: 3)
MLNQNSHFIFIILAIFLVLPLSLLSKNVNAQIPLRRHLLSHESEHYAVIF
DAGSTGSRVHVFRFDEKLGLLPIGNNIEYFMATEPGLSSYAEDPKAAANS
LEPLLDGAEGVVPQELQSETPLELGATAGLRMLKGDAAEKILQAVRNLVK
NQSTFHSKDQWVTILDGTQEGSYMWAAINYLLGNLGKDYKSTTATIDLGG
GSVQMAYAISNEQFAKAPQNEDGEPYVQQKHLMSKDYNLYVHSYLNYGQL
AGRAEIFKASRNESNPCALEGCDGYYSYGGVDYKVKAPKKGSSWKRCRRL
TRHALKINAKCNIEECTFNGVWNGGGGDGQKNIHASSFFYDIGAQVGIVD
TKFPSALAKPIQYLNAAKVACQTNVADIKSIFPKTQDRNIPYLCMDLIYE
YTLLVDGFGLNPHKEITVIHDVQYKNYLVGAAWPLGCAIDLVSSTTNKIR
VASS

Sequence of predicted mature potato apyrase:

(SEQ ID NO: 4)
KNVNAQIPLRRHLLSHESEHYAVIFDAGSTGSRVHVFRFDEKLGLLPIGN
NIEYFMATEPGLSSYAEDPKAAANSLEPLLDGAEGVVPQELQSETPLELG
ATAGLRMLKGDAAEKILQAVRNLVKNQSTFHSKDQWVTILDGTQEGSYMW
AAINYLLGNLGKDYKSTTATIDLGGGSVQMAYAISNEQFAKAPQNEDGEP
YVQQKHLMSKDYNLYVHSYLNYGQLAGRAEIFKASRNESNPCALEGCDGY
YSYGGVDYKVKAPKKGSSWKRCRRLTRHALKINAKCNIEECTFNGVWNGG
GGDGQKNIHASSFFYDIGAQVGIVDTKFPSALAKPIQYLNAAKVACQTNV
ADIKSIFPKTQDRNIPYLCMDLIYEYTLLVDGFGLNPHKEITVIHDVQYK
NYLVGAAWPLGCAIDLVSSTTNKIRVASS

Intestinal-type alkaline phosphatase (Bos taurus) (bold=signal sequence):

(SEQ ID NO: 5)
MQGACVLLLLGLHLQLSLGLVPVEEEDPAFWNRQAAQALDVAKKLQPIQT
AAKNVILFLGDGMGVPTVTATRILKGQMNGKLGPETPLAMDQFPYVALSK
TYNVDRQVPDSAGTATAYLCGVKGNYRTIGVSAAARYNQCKTTRGNEVTS
VMNRAKKAGKSVGVVTTTRVQHASPAGAYAHTVNRNWYSDADLPADAQMN
GCQDIAAQLVNNMDIDVILGGGRKYMFPVGTPDPEYPDDASVNGVRKRKQ
NLVQAWQAKHQGAQYVWNRTALLQAADDSSVTHLMGLFEPADMKYNVQQD
HTKDPTLQEMTEVALRVVSRNPRGFYLFVEGGRIDHGHHDDKAYMALTEA
GMFDNAIAKANELTSELDTLILVTADHSHVFSFGGYTLRGTSIFGLAPSK
ALDSKSYTSILYGNGPGYALGGGSRPDVNDSTSEDPSYQQQAAVPQASET
HGGEDVAVFARGPQAHLVHGVEEETFVAHIMAFAGCVEPYTDCNLPAPTT
ATSIPDAAHLAASPPPLALLAGAMLLLLAPTL

Sequence of predicted mature intestinal-type alkaline phosphatase (Bos taurus):

(SEQ ID NO: 6)
LVPVEEEDPAFWNRQAAQALDVAKKLQPIQTAAKNVILFLGDGMGVPTVT
ATRILKGQMNGKLGPETPLAMDQFPYVALSKTYNVDRQVPDSAGTATAYL
CGVKGNYRTIGVSAAARYNQCKTTRGNEVTSVMNRAKKAGKSVGVVTTTR
VQHASPAGAYAHTVNRNWYSDADLPADAQMNGCQDIAAQLVNNMDIDVIL
GGGRKYMFPVGTPDPEYPDDASVNGVRKRKQNLVQAWQAKHQGAQYVWNR
TALLQAADDSSVTHLMGLFEPADMKYNVQQDHTKDPTLQEMTEVALRVVS
RNPRGFYLFVEGGRIDHGHHDDKAYMALTEAGMFDNAIAKANELTSELDT
LILVTADHSHVFSFGGYTLRGTSIFGLAPSKALDSKSYTSILYGNGPGYA
LGGGSRPDVNDSTSEDPSYQQQAAVPQASETHGGEDVAVFARGPQAHLVH
GVEEETFVAHIMAFAGCVEPYTDCNLPAPTTATSIPDAAHLAASPPPLAL
LAGAMLLLLAPTL

We claim:

1. A method for improving growth of cells cultured in a liquid media comprising culturing the cells in a media comprising one or more enzymes that hydrolyze at least one nucleoside triphosphate, wherein the cells exhibit improved growth compared to cells cultured in media that does not comprise one or more enzymes that hydrolyze at least one nucleoside triphosphate.

2. The method of claim 1, wherein improved growth comprises one or more of greater optical density (OD), higher colony forming units (cfu)/mL, decreased cell lysis, higher proliferation rate, increased cell mass, higher cell numbers, or higher post-culture viability.

3. The method of claim 1 or claim 2, wherein the nucleoside triphosphate comprises one or more of ATP, GTP, CTP, TTP, and/or UTP.

4. The method of claim 3, wherein the nucleoside triphosphate comprises ATP.

5. The method of any one of claims 1-4, wherein the enzyme is one or more phosphatase.

6. The method of claim 5, wherein the phosphatase is one or more of an acid phosphatase, an alkaline phosphatase, or an apyrase.

7. The method of any one of claims 1-6, wherein the cultured cells are bacterial, fungal, archaeal, plant, insect, or mammalian cells.

8. The method of claim 7, wherein the cells are recombinant cells.

9. The method of claim 7 or claim 8, wherein the bacterial cells are gram positive or gram negative.

10. The method of claim 9, wherein the bacterial cells are aerobes or anaerobes.

11. The method of any one of claims 8-10, wherein the bacterial cells are probiotics.

12. The method of claim 7 or claim 8, wherein the fungal cells are filamentous fungal cells.

13. The method of claim 7 or claim 8, wherein the fungal cells are yeast cells.

14. The method of claim 7 or claim 8, wherein the mammalian cells are primary cells, Chinese hamster ovary (CHO) cells or human embryonic kidney (HEK) cells.

15. The method of any one of claims 8-14, wherein the recombinant cells produce an enzyme.

16. The method of any one of claims 8-14, wherein the recombinant cells produce an antibody.

17. The method of any one of claims 14-16, wherein the primary cell is a stem cell or an immune cell.

18. The method of claim 17, wherein the immune cell is selected from the group consisting of: a natural killer (NK) cell, a lymphocyte, a leukocyte, and a monocyte.

19. The method of any one of claims 1-18, wherein the one or more enzymes that hydrolyze at least one nucleoside triphosphate is exogenously added to the media.

20. The method of any one of claims 1-19, wherein the one or more enzymes that hydrolyze at least one nucleoside triphosphate is not recombinantly or naturally produced by cells in the liquid media.

21. The method of any one of claims 1-20, wherein the one or more enzyme that hydrolyzes at least one nucleoside triphosphate comprises one or more enzyme at least about 60% identical to the amino acid sequence of SEQ ID NOs: 1, 2, 3, 4, 5 and/or 6.

22. A method for culturing a probiotic for consumption by a subject, the method comprising culturing probiotic cells in a liquid media comprising one or more enzymes that hydrolyze at least one nucleoside triphosphate, wherein the probiotic cells exhibit improved growth compared to probiotic cells cultured in media that does not comprise one or more enzymes that hydrolyze at least one nucleoside triphosphate.

23. The method of claim 22, wherein improved growth comprises one or more of greater optical density (OD), higher colony forming units (cfu)/mL, decreased cell lysis, higher proliferation rate, increased cell mass, higher cell numbers, or higher post-culture viability.

24. The method of claim 22 or claim 23, wherein the nucleoside triphosphate comprises one or more of ATP, GTP, CTP, TTP, and/or UTP.

25. The method of claim 24, wherein the nucleoside triphosphate comprises ATP.

26. The method of any one of claims 22-25, wherein the enzyme is one or more phosphatase.

27. The method of claim 26, wherein the phosphatase is one or more of an acid phosphatase, an alkaline phosphatase, or an apyrase.

28. The method of any one of claims 22-27, wherein the probiotic cells are bacterial or fungal cells.

29. The method of claim 28, wherein the cells are recombinant cells.

30. The method of claim 28 or claim 29, wherein the bacterial cells are gram positive or gram negative.

31. The method of claim 30, wherein the bacterial cells are aerobes or anaerobes.

32. The method of claim 28 or claim 29, wherein the fungal cells are yeast cells.

33. The method of any one of claims 29-32, wherein the recombinant cells produce an enzyme.

34. The method of any one of claims 33-33, further comprising harvesting the probiotic cells.

35. The method of any one of claims 22-34, further comprising freeze drying or lyophilizing the probiotic cells.

36. The method of any one of claims 22-35, wherein the subject is a human, a companion animal, or livestock.

37. The method of claim 36, wherein the livestock is poultry, swine, or a ruminant animal.

38. The method of claim 36 or claim 37, further comprising formulating the probiotic cells into a dosage form suitable for enteral administration.

39. The method of claim 36 or claim 37, further comprising combining the probiotic cells with an animal feed.

40. The method of claim 36 or claim 37, further comprising administering the probiotic cells to the subject via a waterline.

41. The method of any one of claims 22-40, wherein the one or more enzymes that hydrolyze at least one nucleoside triphosphate is exogenously added to the media.

42. The method of any one of claims 22-41, wherein the one or more enzymes that hydrolyze at least one nucleoside triphosphate is not recombinantly or naturally produced by the probiotic cells in the liquid media.

43. The method of any one of claims 22-42, wherein the one or more enzyme that hydrolyzes at least one nucleoside triphosphate comprises one or more enzyme at least about 60% identical to the amino acid sequence of SEQ ID NOs: 1, 2, 3, 4, 5 and/or 6.

44. A method for producing bacterial cells for use in production of fermented dairy products, the method comprising culturing the bacterial cells in a liquid media comprising one or more enzymes that hydrolyze at least one nucleoside triphosphate, wherein the bacterial cells exhibit improved growth compared to bacterial cells cultured in media that does not comprise one or more enzymes that hydrolyze at least one nucleoside triphosphate.

45. The method of claim 44, wherein improved growth comprises one or more of greater optical density (OD), higher colony forming units (cfu)/mL, decreased cell lysis, higher proliferation rate, increased cell mass, higher cell numbers, or higher post-culture viability.

46. The method of claim 44 or claim 45, wherein the nucleoside triphosphate comprises one or more of ATP, GTP, CTP, TTP, and/or UTP.

47. The method of claim 4464, wherein the nucleoside triphosphate comprises ATP.

48. The method of any one of claims 44-47, wherein the enzyme is one or more phosphatase.

49. The method of claim 48, wherein the phosphatase is one or more of an acid phosphatase, an alkaline phosphatase, or an apyrase.

50. The method of any one of claims 45-49, wherein the bacterial cells are gram positive or gram negative.

51. The method of claim 50, wherein the bacterial cells are thermophilic.

52. The method of claim 50 or claim 51, wherein the bacterial cells are selected from the group consisting of a Lactobacillus spp., a Streptococcus spp., and a Lactococcus spp.

53. The method of any one of claims 44-52, wherein the fermented dairy product is a yoghurt, a cream, a matured cream, a cheese, fromage frais, a milk beverage, a processed cheese, a cream dessert, a cottage cheese, an infant milk, or a kefir.

54. The method of any one of claims 44-53, wherein the one or more enzymes that hydrolyze at least one nucleoside triphosphate is exogenously added to the media.

55. The method of any one of claims 44-54, wherein the one or more enzymes that hydrolyze at least one nucleoside triphosphate is not recombinantly or naturally produced by the bacterial cells in the liquid media.

56. The method of any one of claims 44-55, wherein the one or more enzyme that hydrolyzes at least one nucleoside triphosphate comprises one or more enzyme at least about 60% identical to the amino acid sequence of SEQ ID NOs: 1, 2, 3, 4, 5 and/or 6.

57. A cell culture media comprising one or more enzymes that hydrolyze at least one nucleoside triphosphate, wherein said one or more enzymes that hydrolyze at least one nucleoside triphosphate is a) recombinantly produced; and b) exogenously added to the media.

58. The media of claim 57, wherein the media does not comprise one or more cells.

59. The media of claim 57, wherein the media comprises one or more cells and wherein said one or more cells do not recombinantly produce one or more enzymes that hydrolyze at least one nucleoside triphosphate.

60. The media of any one of claims 57-59, wherein the nucleoside triphosphate comprises one or more of ATP, GTP, CTP, TTP, and/or UTP.

61. The media of claim 60, wherein the nucleoside triphosphate comprises ATP.

62. The media of any one of claims 57-61, wherein the enzyme is one or more phosphatase.

63. The media of claim 60, wherein the phosphatase is one or more of an acid phosphatase, an alkaline phosphatase, or an apyrase.

64. The media of any one of claims 59-63, wherein the cells are bacterial, fungal, archaeal, plant, insect, or mammalian cells.

65. The media of claim 64, wherein the cells are recombinant cells.

66. The media of claim 64 or claim 65, wherein the bacterial cells are gram positive or gram negative.

67. The media of claim 64 or claim 65, wherein the bacterial cells are aerobes or anaerobes.

68. The media of any one of claims 64-67, wherein the bacterial cells are probiotics.

69. The media of claim 64 or claim 65, wherein the fungal cells are filamentous fungal cells.

70. The media of claim 64 or claim 65, wherein the fungal cells are yeast cells.

71. The media of claim 64 or claim 65, wherein the mammalian cells are primary cells, Chinese hamster ovary (CHO) cells or human embryonic kidney (HEK) cells.

72. The media of any one of claims 65-71, wherein the recombinant cells produce an enzyme.

73. The media of any one of claims 65-71, wherein the recombinant cells produce an antibody.

74. The media of any one of claims 71-73, wherein the primary cell is a stem cell or an immune cell.

75. The media of claim 74, wherein the immune cell is selected from the group consisting of: a natural killer (NK) cell, a lymphocyte, a leukocyte, and a monocyte.

76. The media of any one of claims 57-75, wherein the one or more enzyme that hydrolyzes at least one nucleoside triphosphate comprises one or more enzyme at least about 60% identical to the amino acid sequence of SEQ ID NOs: 1, 2, 3, 4, 5 and/or 6.

77. A method for producing a liquid cell culture media comprising combining a cell culture media with one or more recombinant enzymes that hydrolyze at least one nucleoside triphosphate.

78. The method of claim 77, wherein the nucleoside triphosphate comprises one or more of ATP, GTP, CTP, TTP, and/or UTP.

79. The method of claim 78, wherein the nucleoside triphosphate comprises ATP.

80. The method of any one of claims 77-79, wherein the enzyme is one or more phosphatase.

81. The method of claim 80, wherein the phosphatase is one or more of an acid phosphatase, an alkaline phosphatase, or an apyrase.

82. The method of any one of claims 77-81, wherein the one or more recombinant enzyme that hydrolyzes at least one nucleoside triphosphate comprises one or more enzyme at least about 60% identical to the amino acid sequence of SEQ ID NOs: 1, 2, 3, 4, 5 and/or 6.

83. A method for improving the growth of cells engineered to produce a first recombinant protein comprising culturing the cell in a media comprising one or more enzymes that hydrolyze at least one nucleoside triphosphate, wherein 1) the cells exhibit improved growth compared to cells cultured in media that does not comprise one or more enzymes that hydrolyze at least one nucleoside triphosphate; and 2) wherein the one or more enzymes that hydrolyze at least one nucleoside triphosphate is a) exogenously added to the media; and/or b) recombinantly expressed by the engineered cells in addition to the first recombinant protein.

84. The method of claim 83, wherein improved growth comprises one or more of greater optical density (OD), higher colony forming units (cfu)/mL, decreased cell lysis, higher proliferation rate, increased cell mass, higher cell numbers, or higher first recombinant protein production.

85. The method of claim 83 or claim 84, wherein the nucleoside triphosphate comprises one or more of ATP, GTP, CTP, TTP, and/or UTP.

86. The method of claim 85, wherein the nucleoside triphosphate comprises ATP.

87. The method of any one of claims 83-86, wherein the enzyme is one or more phosphatase.

88. The method of claim 87, wherein the phosphatase is one or more of an acid phosphatase, an alkaline phosphatase, or an apyrase.

89. The method of any one of claims 83-88, wherein the cultured cells are bacterial, fungal, archaeal, plant, insect, or mammalian cells.

90. The method of claim 89, wherein the bacterial cells are gram positive or gram negative.

91. The method of claim 89, wherein the bacterial cells are aerobes or anaerobes.

92. The method of claim 89, wherein the fungal cells are filamentous fungal cells.

93. The method of claim 89, wherein the fungal cells are yeast cells.

94. The method of claim 89, wherein the mammalian cells are primary cells, Chinese hamster ovary (CHO) cells or human embryonic kidney (HEK) cells.

95. The method of any one of claims 83-94, wherein the recombinant cells produce an enzyme.

96. The method of any one of claims 83-94, wherein the recombinant cells produce an antibody.

97. The method of any one of claims 94-96, wherein the primary cell is a stem cell or an immune cell.

98. The method of claim 97, wherein the immune cell is selected from the group consisting of: a natural killer (NK) cell, a lymphocyte, a leukocyte, and a monocyte.

99. The method of any one of claims 83-98, wherein one or more polynucleotides encoding the one or more enzymes that hydrolyze at least one nucleoside triphosphate is recombinantly expressed on one or more extrachromosomal vector in the cell.

100. The method of any one of claims 83-98, wherein one or more polynucleotides encoding the one or more enzymes that hydrolyze at least one nucleoside triphosphate is recombinantly expressed via stable integration into the chromosome of the cell.

101. The method of any one of claims 83-100, wherein the one or more enzymes that hydrolyze at least one nucleoside triphosphate is recombinantly expressed by the engineered cells in addition to the first recombinant protein and is further engineered to be secreted by the cell.

102. The method of any one of claims 83-101, wherein the one or more recombinant enzyme that hydrolyzes at least one nucleoside triphosphate comprises one or more enzyme at least about 60% identical to the amino acid sequence of SEQ ID NOs: 1, 2, 3, 4, 5 and/or 6.


SEQUENCE LISTING (6 sequences)

SEQ ID NO: 1 - protein, 388 aa, Gallaecimonas xiamenensis
ADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLAPSVGDGRPEGRPGQQPGGVQHRQH

SEQ ID NO: 2 - protein, 391 aa, Gallaecimonas xiamenensis
AGKADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLAPSVGDGRPEGRPGQQPGGVQHRQH

SEQ ID NO: 3 - protein, 454 aa, Solanum tuberosum
MLNQNSHFIFIILAIFLVLPLSLLSKNVNAQIPLRRHLLSHESEHYAVIFDAGSTGSRVHVFRFDEKLGLLPIGNNIEYFMATEPGLSSYAEDPKAAANSLEPLLDGAEGVVPQELQSETPLELGATAGLRMLKGDAAEKILQAVRNLVKNQSTFHSKDQWVTILDGTQEGSYMWAAINYLLGNLGKDYKSTTATIDLGGGSVQMAYAISNEQFAKAPQNEDGEPYVQQKHLMSKDYNLYVHSYLNYGQLAGRAEIFKASRNESNPCALEGCDGYYSYGGVDYKVKAPKKGSSWKRCRRLTRHALKINAKCNIEECTFNGVWNGGGGDGQKNIHASSFFYDIGAQVGIVDTKFPSALAKPIQYLNAAKVACQTNVADIKSIFPKTQDRNIPYLCMDLIYEYTLLVDGFGLNPHKEITVIHDVQYKNYLVGAAWPLGCAIDLVSSTTNKIRVASS

SEQ ID NO: 4 - protein, 429 aa, Solanum tuberosum
KNVNAQIPLRRHLLSHESEHYAVIFDAGSTGSRVHVFRFDEKLGLLPIGNNIEYFMATEPGLSSYAEDPKAAANSLEPLLDGAEGVVPQELQSETPLELGATAGLRMLKGDAAEKILQAVRNLVKNQSTFHSKDQWVTILDGTQEGSYMWAAINYLLGNLGKDYKSTTATIDLGGGSVQMAYAISNEQFAKAPQNEDGEPYVQQKHLMSKDYNLYVHSYLNYGQLAGRAEIFKASRNESNPCALEGCDGYYSYGGVDYKVKAPKKGSSWKRCRRLTRHALKINAKCNIEECTFNGVWNGGGGDGQKNIHASSFFYDIGAQVGIVDTKFPSALAKPIQYLNAAKVACQTNVADIKSIFPKTQDRNIPYLCMDLIYEYTLLVDGFGLNPHKEITVIHDVQYKNYLVGAAWPLGCAIDLVSSTTNKIRVASS

SEQ ID NO: 5 - protein, 532 aa, Bos taurus
MQGACVLLLLGLHLQLSLGLVPVEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCKTTRGNEVTSVMNRAKKAGKSVGVVTTTRVQHASPAGAYAHTVNRNWYSDADLPADAQMNGCQDIAAQLVNNMDIDVILGGGRKYMFPVGTPDPEYPDDASVNGVRKRKQNLVQAWQAKHQGAQYVWNRTALLQAADDSSVTHLMGLFEPADMKYNVQQDHTKDPTLQEMTEVALRVVSRNPRGFYLFVEGGRIDHGHHDDKAYMALTEAGMFDNAIAKANELTSELDTLILVTADHSHVFSFGGYTLRGTSIFGLAPSKALDSKSYTSILYGNGPGYALGGGSRPDVNDSTSEDPSYQQQAAVPQASETHGGEDVAVFARGPQAHLVHGVEEETFVAHIMAFAGCVEPYTDCNLPAPTTATSIPDAAHLAASPPPLALLAGAMLLLLAPTL

SEQ ID NO: 6 - protein, 513 aa, Bos taurus
LVPVEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCKTTRGNEVTSVMNRAKKAGKSVGVVTTTRVQHASPAGAYAHTVNRNWYSDADLPADAQMNGCQDIAAQLVNNMDIDVILGGGRKYMFPVGTPDPEYPDDASVNGVRKRKQNLVQAWQAKHQGAQYVWNRTALLQAADDSSVTHLMGLFEPADMKYNVQQDHTKDPTLQEMTEVALRVVSRNPRGFYLFVEGGRIDHGHHDDKAYMALTEAGMFDNAIAKANELTSELDTLILVTADHSHVFSFGGYTLRGTSIFGLAPSKALDSKSYTSILYGNGPGYALGGGSRPDVNDSTSEDPSYQQQAAVPQASETHGGEDVAVFARGPQAHLVHGVEEETFVAHIMAFAGCVEPYTDCNLPAPTTATSIPDAAHLAASPPPLALLAGAMLLLLAPTL